(19) United States
(12) Patent Application Publication
(10) Pub. No.: US 2017/0354425 A1
(43) Pub. Date: Dec. 14, 2017

(54) SURGICAL MEASUREMENT INSTRUMENT

(71) Applicant: KYOCERA CORPORATION, Kyoto-shi, Kyoto (JP)

(72) Inventors: Hironori ZAIMA, Kyoto-shi (JP); Masahiko HASHIDA, Kyoto-shi (JP)

(73) Assignee: Kyocera Corporation, Kyoto-shi, Kyoto (JP)

(21) Appl. No.: 15/546,901

(22) PCT Filed: Nov. 20, 2015
(86) PCT No.: PCT/JP2015/082772

§ 371 Date: Jul. 27, 2017

(30) Foreign Application Priority Data
Jan. 27, 2015 (JP) 2015-013310

(51) Int. Cl. A61B 17/17; A61B 17/14; A61B 17/16; A61F 2/46; A61B 17/15

(52) U.S. Cl. CPC A61B 17/17; A61F 2/4657; A61B 17/155; A61B 17/1671; A61F 2/461; A61B 17/1675; A61B 17/14; A61F 2/4611; A61B 17/157

(57) ABSTRACT

A surgical measurement instrument for use in surgery is configured so that a measurement operation can be more readily performed using this surgical measurement instrument. The surgical measurement instrument has a laser application portion. The laser application portion is used in surgery to perform treatment on a tibia of a patient. The laser application portion can apply a laser beam to measure a positional relationship between the tibia and a knee joint center.

### TECHNICAL FIELD

The present invention relates to surgical measurement instruments used when performing treatment on a bone of a patient.

### BACKGROUND ART

For example, in spine correction surgery to correct spine distortion, an operation to connect nut members that are screwed into a plurality of vertebrae to one correction rod is performed (e.g. see Non-Patent Document 1). Due to the plurality of nut members entering a state of being connected to the correction rod, the nut members push the respective vertebrae toward the rod. As a result, the spine having the plurality of vertebrae is corrected so as to be straightened when seen from the back side of the patient.

In the above-described spine correction surgery, the correction rod connected to the plurality of nut members needs to be arranged parallel to the up-down direction of the patient (the direction in which the backbone extends). For this purpose, for example, it is conceivable to prepare a measurement rod to measure the direction in which the correction rod is arranged, in addition to the correction rod, and measure the direction of the correction rod using this measurement rod.

### CITATION LIST

### Patent Document

Patent Application Document 1: JP 5-505749A

Non-Patent Document 1: http://www.implamed.com.tr/medicrea_pass.php

### DISCLOSURE OF THE INVENTION

### Problem to be Solved by the Invention

However, if the measurement operation is performed using the measurement rod, a surgeon needs to keep holding the measurement rod, which is an elongated and heavy item, and this is troublesome.

There are also similar problems in other surgeries to perform treatment on a bone of a patient, particularly in artificial joint replacement surgery, in which the mutual positional relationship between a plurality of bones needs to be adjusted.

In view of the foregoing situation, an object of the present invention is to enable a measurement operation to be more readily performed using a surgical measurement instrument used in surgery.

### Means for Solving the Problem

(1) A surgical measurement instrument according to the present invention to achieve the above-stated object is a surgical measurement instrument used in surgery to perform treatment on a bone of a patient, including: a laser application portion capable of applying a laser beam for measuring a positional relationship concerning the bone.

With this configuration, the laser application portion is configured to apply a laser beam to measure the positional relationship concerning a bone of the patient. This configuration does not require a surgeon to hold a heavy item such as a measurement rod in order to measure the positional relationship concerning a bone of the patient. Accordingly, the burden on the surgeon when measuring the positional relationship concerning a bone of the patient can be reduced. In addition, a laser beam can be formed in a thinner line than the measurement rod. Accordingly, the laser beam can be more readily and accurately applied to a predetermined portion of the patient. As a result, with the surgical measurement instrument according to the present invention, used in surgery, a measurement operation can be more readily performed using this surgical measurement instrument.

(2) Preferably, the laser application portion is configured to apply the laser beam to measure relative positions between a plurality of bones of the patient.

With this configuration, for example, in surgery to correct relative positions between a plurality of bones of the patient, the relative positions between the plurality of bones can be more readily measured. For example, in corrective surgery to treat scoliosis, i.e. an unnaturally curved vertebral column of the patient, the surgical measurement instrument can be used to measure the alignment direction of the spine.

(3) Preferably, the laser application portion is configured to apply the laser beam to measure a positional relationship between the bone of the patient and an instrument to be used in implant placement surgery to install a predetermined implant on the bone of the patient.

With this configuration, in implant placement surgery, the surgeon can more readily measure the positional relationship between the instrument and the bone of the patient.

(4) More preferably, the instrument is a jig for installing the implant in a body of the patient.

With this configuration, for example, in the case of temporarily placing, on the bone of the patient, the jig to be used in implant placement surgery, the surgeon can more readily measure the relative positions between this bone of the patient and the jig.

(5) More preferably, the implant placement surgery includes artificial leg joint implant placement surgery for installing the implant on a leg joint including a distal part of a tibia serving as the bone of the patient, the jig includes a tibia distal part cutting guide to be used when cutting the distal part to install the implant on the distal part, and the laser application portion is configured to apply the laser beam toward a knee joint center of the patient, in a state of being supported by the tibia distal part cutting guide.

With this configuration, in artificial leg joint implant placement surgery, the surgeon can more readily measure the position of the implant to be installed in the distal part of the tibia of the patient and the position of the knee joint center. In addition, the laser application portion is installed on the tibia distal part cutting guide portion. With this configuration, the laser application portion is held in a stable orientation by the distal part of the tibia. Accordingly, the surgeon can more accurately measure positions using the laser beam in a state where the laser beam position is less likely to shift, unlike in the case of a configuration in which, when the surgeon uses a measurement rod instead of the laser application portion and holds this measurement rod, the position of this measurement rod is likely to shift.

(6) Preferably, the surgery includes spine correction surgery for correcting a spine of the patient, the surgical measurement instrument further comprises a fixed jig that is to be fixed to a pelvis of the patient, and the laser application portion is configured to apply the laser beam to measure a plurality of vertebrae of the spine, in a state of being supported by the fixed jig.

With this configuration, in spine correction surgery, the alignment direction of the spine, for example, can be more readily measured by surgeon. In addition, the laser application portion is installed on the fixed jig. Thus, the laser application portion is held in a stable orientation by the fixed jig. Accordingly, the surgeon can more accurately measure positions using the laser beam in a state where the laser beam position is less likely to shift, unlike in the case of a configuration in which, when the surgeon uses a measurement rod instead of the laser application portion and holds this measurement rod, the position of this measurement rod is likely to shift.

(7) Preferably, the implant placement surgery includes artificial knee joint implant placement surgery for installing the implant on a knee joint including a distal part of a femur serving as the bone of the patient, the jig includes a guide member holder portion for holding a guide member that guides a bone-cutting position when cutting a bone in a proximal part of a tibia of the patient, the guide member holder portion includes a proximal side portion to be connected to the proximal part of the tibia, a distal side portion to be connected to the distal part of the tibia, and a rod for connecting the proximal side portion and the distal side portion to each other, and the laser application portion is configured to apply the laser beam to measure parallelism between the rod and the tibia, in a state of being supported by the guide member holder portion.

With this configuration, in artificial knee joint implant placement surgery, the surgeon can more readily measure the parallelism between the rod of the guide member holder portion and the tibia. In addition, the laser application portion is installed on the guide member holder portion. Thus, the laser application portion is held in a stable orientation by the guide member holder portion. Accordingly, the surgeon can more accurately measure positions using the laser beam in a state where the laser beam position is less likely to shift, unlike in the case of a configuration in which, when the surgeon uses a measurement rod instead of the laser application portion and holds this measurement rod, the position of this measurement rod is likely to shift.

(8) More preferably, the laser application portion is configured to apply the laser beam to measure the bone-cutting position in the proximal part of the tibia.

With this configuration, in artificial knee joint implant placement surgery, the surgeon can more readily and accurately measure the bone-cutting position in the proximal part of the tibia.

(9) Preferably, the implant placement surgery includes artificial knee joint implant placement surgery for installing the implant on a knee joint including a distal part of a femur serving as the bone of the patient, the jig includes a drill for forming a reamer hole in the distal part of the femur, and the laser application portion is configured to apply the laser beam to measure a coaxiality between the drill and the femur, in a state of being supported by the drill.

With this configuration, in artificial knee joint implant placement surgery, the surgeon can more readily measure the coaxiality between the distal part of the femur and the drill. In addition, the laser application portion is installed on the drill. Thus, the laser application portion is held in a stable orientation by the drill. As a result, the surgeon can more accurately measure the positions using the laser beam in a state where the laser beam position is less likely to shift.

(10) Preferably, the implant placement surgery includes artificial knee joint implant placement surgery for installing the implant on a knee joint including a distal part of a femur serving as the bone of the patient, the jig includes a valgus alignment guide to be attached to the femur to guide insertion of a predetermined medullary cavity rod into a medullary cavity portion of the femur, the valgus alignment guide being for adjusting a position of the medullary cavity rod in a valgus angle direction of the femur, and the laser application portion is configured to apply the laser beam to measure a positional relationship between the valgus alignment guide and the femur, in a state of being supported by the valgus alignment guide.

With this configuration, in artificial knee joint implant placement surgery, the surgeon can more readily measure the positional relationship between the distal part of the femur and the valgus alignment guide. In addition, the laser application portion is installed in the valgus alignment guide. Thus, the laser application portion is held in a stable orientation by the valgus alignment guide. Accordingly, the surgeon can more accurately measure positions using the laser beam in a state where the laser beam position is less likely to shift, unlike in the case of a configuration in which, when the surgeon uses a measurement rod instead of the laser application portion and holds this measurement rod, the position of this measurement rod is likely to shift.

(11) More preferably, the laser application portion is configured to apply the laser beam to indicate a bone head center of the femur, in a state of being supported by the valgus alignment guide.

With this configuration, the surgeon can more readily measure the positional relationship between the bone head center of the femur and the valgus alignment guide. In addition, the laser application portion is held in a stable orientation by the valgus alignment guide. Accordingly, the surgeon can more accurately measure positions using the laser beam in a state where the laser beam position is less likely to shift, unlike in the case of a configuration in which, when the surgeon uses a measurement rod instead of the laser application portion and holds this measurement rod, the position of this measurement rod is likely to shift.

(12) Preferably, the implant placement surgery includes artificial knee joint implant placement surgery for installing the implant on a knee joint including a distal part of a femur serving as the bone of the patient, the jig includes a spacer that is to be arranged between a cut-bone face formed in the distal part of the femur and a cut-bone face formed in a proximal part of a tibia of the patient, and the laser application portion is configured to apply the laser beam to indicate a bone head center of the femur and a leg joint center of the patient, in a state of being supported by the spacer.

With this configuration, when checking, for example, that the knee joint center, the bone head center of the femur, and the leg joint center are arranged in a straight line (alignment), the surgeon can more readily measure the alignment using the laser beam as a mark. In addition, the laser application portion is held in a stable orientation by the spacer. Accordingly, the surgeon can more accurately measure positions using the laser beam in a state where the laser beam position is less likely to shift, unlike in the case of a configuration in which, when the surgeon uses a measurement rod instead of the laser application portion and holds this measurement rod, the position of this measurement rod is likely to shift.

(13) Preferably, the implant placement surgery includes artificial knee joint implant placement surgery for installing the implant on a knee joint including a distal part of a femur serving as the bone of the patient, the jig includes a sizer member for positioning a pin that is to be driven into a cut-bone face formed in the distal part of the femur, and the laser application portion is configured to apply the laser beam to measure a positional relationship between the cut-bone face and the sizer member, in a state of being supported by the sizer member.

With this configuration, the surgeon can more readily measure the positional relationship between the cut-bone face and the sizer member. In addition, the laser application portion is held in a stable orientation by the sizer member. As a result, the surgeon can more accurately measure the positions using the laser beam in a state where the laser beam position is less likely to shift.

(14) Preferably, the implant placement surgery includes artificial knee joint implant placement surgery for installing the implant on a knee joint including a distal part of a femur serving as the bone of the patient, the jig includes a guide member that is to be installed in a cut-bone face formed in the distal part of the femur, the guide member being for guiding a cutter for forming an additional cut-bone face in the distal part, and the laser application portion is configured to apply the laser beam to measure a positional relationship between the guide member and the distal part, in a state of being supported by the guide member.

With this configuration, the surgeon can more readily measure the positional relationship between the cut-bone face and the guide member. In addition, the laser application portion is held in a stable orientation by the guide member. As a result, the surgeon can more accurately measure the positions using the laser beam in a state where the laser beam position is less likely to shift.

(15) Preferably, the laser application portion is configured to radially apply the laser beam to the patient.

With this configuration, the laser beam is applied to more portions. Thus, the surgeon can more readily visually check the positional relationship between each portion to which the laser beam is applied and a reference portion.

### Effects of the Invention

With the surgical measurement instrument according to the present invention, used in surgery, a measurement operation can be more readily performed using this surgical measurement instrument.

### BRIEF DESCRIPTION OF DRAWINGS

FIG. 1 is a side view showing a surgical measurement instrument 1 according to a first embodiment of the present invention, a portion of the skeleton of a patient, and the like.

FIG. 2 is a front elevational view showing the surgical measurement instrument 1, a portion of the skeleton of a patient, and the like.

FIG. 3 is a flowchart showing the main points of an exemplary flow of artificial leg joint implant placement surgery.

FIG. 4 is a rear view showing a measurement instrument 23 according to a second embodiment of the present invention and a portion of the skeleton of a patient.

FIG. 5 is a rear view showing a state where the spine has been corrected.

FIG. 6 is a flowchart showing the main points of an exemplary flow of spine correction surgery.

FIG. 7 is a diagram illustrating a third embodiment of the present invention, and is a side view with a partial cross section showing a state where an artificial knee joint implant has been installed in a patient.

FIG. 8 is a perspective view showing a state where a surgical device 40 has been attached to a tibia.

FIG. 9 is a perspective view showing a surgical device 55 and the like.

FIG. 10 is a perspective view showing the main points to illustrate a procedure for forming a reamer hole in a distal part of a femur.

FIG. 11 is a perspective view showing the main points to illustrate a procedure for inserting a medullary cavity rod in a distal part of a femur.

FIG. 12 is a front elevational view showing a surgical device 78 and the like.

FIG. 13 is a front elevational view illustrating a procedure for checking a gap between a main face of a cut-bone face of a femur and a cut-bone face of a tibia.

FIG. 14 shows a modification of a measurement instrument 81 to be attached to a spacer.

FIG. 15 is a perspective view showing the main points to illustrate a procedure for fixing a sizer member to a distal part of a femur.

FIG. 16 is a side view showing the main points to illustrate a procedure for installing a guide member 122 in a distal part of a femur.

### DESCRIPTION OF EMBODIMENTS

Hereinafter, modes for implementing the present invention will be described with reference to the drawings. The present invention can be widely applied as a surgical measurement instrument.

### First Embodiment

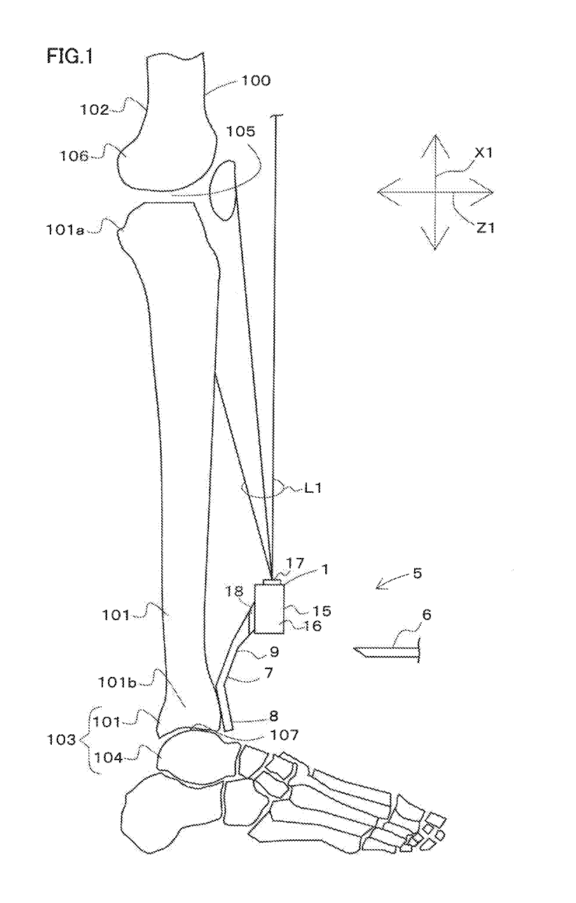

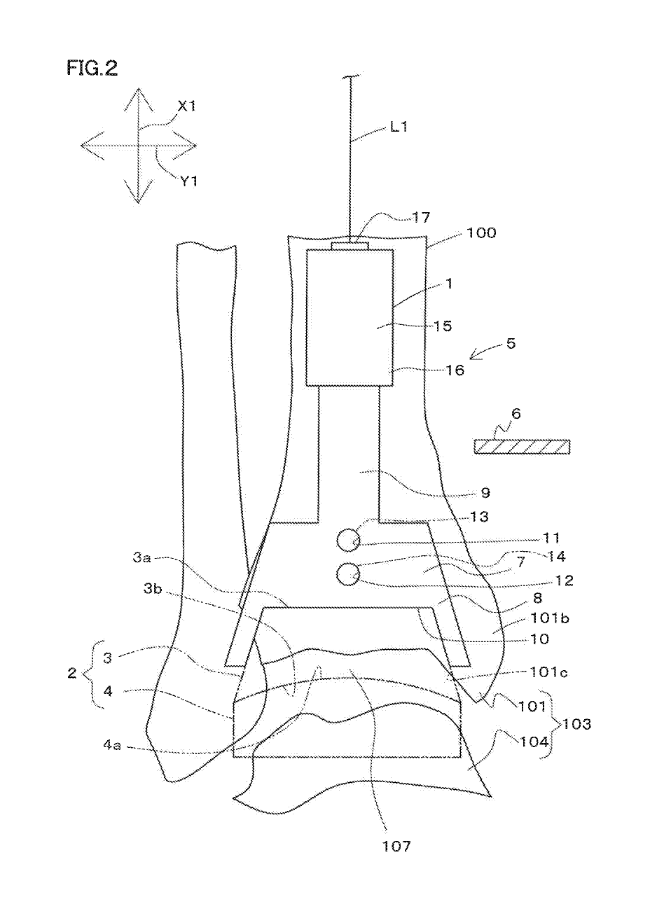

FIG. 1 is a side view showing a surgical measurement instrument 1 according to the first embodiment of the present invention, a portion of the skeleton of a patient 100, and the like. FIG. 2 is a front elevational view showing the surgical measurement instrument 1, a portion of the skeleton of the patient 100, and the like.

This embodiment will describe, with reference to FIGS. 1 and 2, the main points regarding artificial leg joint implant placement surgery, which is implant placement surgery, i.e. surgery to perform treatment on a bone of the patient 100. The artificial leg joint implant placement surgery is surgery to replace a leg joint 103, which includes a distal part 101b of a tibia 101 of the patient 100, with an artificial leg joint implant 2. Note that, although FIGS. 1 and 2 show the skeleton of the patient 100, artificial leg joint implant placement surgery is performed with an incision made only in the periphery of the leg joint 103 of the patient 100.

Note that, in the following description, “front” and “rear” refer to the front and rear of the patient in a standing state. “Above/upper” and “below/lower” refer to the above/upper and below/lower of the patient in a standing state. “Left” and “right” refer to the left and right of the patient. The surgical measurement instrument 1 will be described based on a reference state where the surgical measurement instrument 1 is installed in the patient 100.

The artificial leg joint implant 2 has a tibia component 3, which is to be fixed to the distal part 10b of the tibia 101 of the patient 100, and a talus component 4, which is to be fixed to a talus 104 and can be displaced relative to the tibia component 3.

The tibia component 3 is formed in a block shape. The tibia component 3 has, when seen from the front, a fixing portion 3a, which is formed in a trapezoid shape, and a slide face 3b, which faces the talus component 4 side. An outer face of the fixing portion 3a is fixed to a cut-bone face 101c, which is formed in the distal part 101b of the tibia 101. The talus component 4 is formed in a block shape. The talus component 4 is fixed to the talus 104. The talus component 4 has a slide face 4a. The slide faces 4a and 3b are formed to be curved. The slide face 4a is in slidable contact with the slide face 3b, and cooperates with the slide face 3b to form a joint. With this configuration, as a result of the slide faces 3b and 4a sliding against each other, the talus 104 is displaced relative to the tibia 101.

The aforementioned tibia component 3 is fixed to the cut-bone face 101c of the tibia 101, as mentioned above. This cut-bone face 101c is formed by a surgeon using a surgical device 5.

The surgical device 5 has a cutter 6 to cut a portion of the distal part 101b of the tibia 101, a tibia distal part cutting guide 7, and the surgical measurement instrument 1.

The tibia distal part cutting guide 7 is an example of an “instrument” according to the present invention, and is also an example of a “jig”. The tibia distal part cutting guide 7 is used when cutting the distal part 101b in order to install the tibia component 3 on the distal part 101b of the tibia 101. The tibia distal part cutting guide 7 is a Y-shaped member that is formed using a plate-shaped member. The tibia distal part cutting guide 7 is formed in a substantially V-shape when seen from the side.

The tibia distal part cutting guide 7 has a guide body 8 and an extension portion 9.

The guide body 8 is a portion that is to be placed along the distal part 101b of the tibia 101, and is formed in a substantially U-shape. A guide face 10 is formed in the guide body 8. The guide face 10 is formed in a shape that matches the shape of the outer face of the fixing portion 3a of the tibia component 3. The guide face 10 is a portion that is to be placed along the distal part 101b when the surgeon forms the fixing portion 3a in the distal part 101b of the tibia 101 using the cutter 6. The surgeon forms the fixing portion 3a in the distal part 101b by moving the cutter 6 along this guide face 10.

Fixing pin holes 11 and 12 are formed in the guide body 8. These fixing pin holes 11 and 12 are arranged substantially at the center of the guide body 8, and are arranged in the longitudinal direction of the tibia distal part cutting guide 7. Corresponding fixing pins 13 and 14 are inserted into the respective fixing pins 11 and 12. These fixing pins 13 and 14 are fixed to the distal part 101b of the tibia 101. Thus, the tibia distal part cutting guide 7 is fixed to the tibia 101. The extension portion 9 extends from the guide body 8.

The extension portion 9 is a portion that extends away from the tibia 101 as it extends away from the guide body 8. The surgical measurement instrument (hereinafter also referred to simply as a measurement instrument) 1 is installed on this extension portion 9.

The measurement instrument 1 has a laser application portion 15 and a connecting portion 18.

The laser application portion 15 is configured to apply a laser beam to measure a positional relationship concerning the skeleton of the patient 100. In this embodiment, the laser application portion 15 is configured to apply a laser beam L1 in order to measure the positional relationship between a knee joint center 105 of the patient 100 and the tibia distal part cutting guide 7 that is used in artificial leg joint implant placement surgery to install the tibia component 3 on the tibia 101.

The laser application portion 15 has a configuration in which, for example, a battery and a laser beam source (not shown) are housed in a casing 16, which is made of a synthetic resin. The laser beam L1 is applied from an application face 17, which is formed in a side face of the casing 16 of the laser application portion 15. The laser application portion 15 is installed on the extension portion 9 so that the laser beam L1 extends in a direction that coincides with the longitudinal direction of the extension portion 9. The laser application portion 15 is supported by the extension portion 9 via the connecting portion 18, and the application face 17 of the laser application portion 15 faces the knee joint center 105 side of the patient 100.

The connecting portion 18 is provided in order to connect the laser application portion 15 to the tibia distal part cutting guide 7. The connecting portion 18 is a plate-shaped attachment member, for example. The extension portion 18 is fixed to, for example, a leading end of the extension portion 9, and is also fixed to the casing 16 of the laser application portion 15. The laser application portion 15 is arranged so as to apply the laser beam L1 toward the knee joint center 105 of the patient 100, in a state of being supported by the tibia distal part cutting guide 7 via the connecting portion 18.

Specifically, as a result of the surgeon adjusting the position of the tibia distal part cutting guide 7 relative to the tibia 101, the laser beam L1 is set to pass through the knee joint center 105 when seen in plan view. Note that “seen from the front” refers to a visual point of the surgeon in a state of facing the front of the patient 100. “Seen from the side” refers to a visual point of the surgeon in a state of facing the right side or left side of the patient 100. The laser application portion 15 is configured to radially emit the laser beam L1 when seen from the side. Thus, the laser beam L1 is applied to a plurality of portions on the skin surface of the leg of the patient 100.

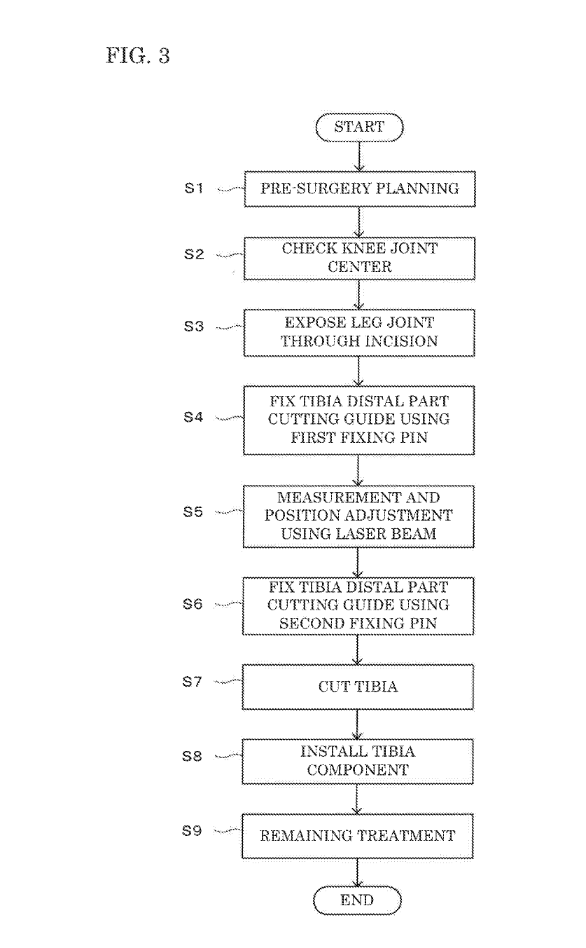

Next, an overview of a surgical procedure for artificial leg joint implant placement surgery will be described. FIG. 3 is a flowchart showing the main points of an exemplary flow of artificial leg joint implant placement surgery. Note that, when a description is given with reference to a flowchart, diagrams other than the flowchart will also be referred to as appropriate.

Referring to FIG. 3, in artificial leg joint implant placement surgery, first, pre-surgery planning is carried out (step S1). In the pre-surgery planning, first, the lower half of the body of the patient 100 that includes an affected area of the patient 100 and the surrounding portion of the affected area is subjected to X-ray imaging or CT imaging. The surgeon then determines the size of the artificial leg joint implant 2 based on images obtained through X-ray imaging or CT imaging.

Next, the surgeon begins surgery. Specifically, the surgeon visually checks the position of the knee joint center 105 (step S2). Next, the surgeon makes an incision in an area near the leg joint 103 of the patient 100 from the front side of the patient 100, and exposes the distal part 101b of the tibia 101 (step S3). Next, the surgeon fixes the tibia distal part cutting guide 7, to which the laser application portion 15 has been fixed, to the distal part 101b of the tibia 101 using one fixing pin (fixing pin 13 or fixing pin 14) (step S4). At this time, the surgeon appropriately sets the distance between the distal part 101b of the tibia 101 and the guide face 10 in accordance with the size of the tibia component 3 that is to be installed in the distal part 101b of the tibia 101.

Next, the surgeon performs a measurement operation and an operation to adjust the position of the tibia distal part cutting guide 7, using the laser beam L applied from the laser application portion 15 (step S5). More specifically, the surgeon, in a state of looking at the patient 100 from the front, adjusts the orientation of the laser application portion 15 (tibia distal part cutting guide 7) so that the laser beam L overlaps the knee joint center 105. Next, the surgeon fixes the tibia distal part cutting guide 7 to the distal part 101b of the tibia 101 using the other fixing pin (fixing pin 13 or fixing pin 14) (step S6). Thus, the tibia distal part cutting guide 7 is fixed to the tibia 101.

Next, the surgeon moves the cutter 6 along the guide face 10 of the tibia distal part cutting guide 7, thereby cutting the distal part 101b of the tibia 101 into a shape that simulates the shape of the guide face 10 (step S7). Thus, the fixing portion 3a is formed on the distal part 101b of the tibia 101. Thereafter, the surgeon removes the tibia distal part cutting guide 7 from the tibia 101. The tibia component 3 is fixed to the fixing portion 3a of the tibia 101 (step S8). Thereafter, the surgeon performs the remaining treatment, such as an operation to attach the talus component 4 to the talus 104 and stitch the incision area near the leg joint 103 (step S9).

As described above, with the surgical measurement instrument 1, the laser application portion 15 is configured to apply the laser beam L1 to measure the positional relationship concerning the tibia 101 of the patient 100. With this configuration, the surgeon does not need to hold a heavy item, such as a measurement rod, in order to measure the positional relationship concerning the tibia 101 of the patient. Accordingly, the burden on the surgeon when measuring the positional relationship concerning the tibia 101 of the patient 100 can be reduced. In addition, the laser beam L1 can be formed as a thinner line than a measurement rod. Accordingly, the laser beam L1 can be more readily and accurately applied to the knee joint center 105 of the patient 100. As a result, a measurement operation can be more readily performed using the measurement instrument 1.

The measurement instrument 1 is configured to apply the laser beam L1 in order to measure the positional relationship between the tibia 101 of the patient 100 and the tibia distal part cutting guide 7 that is used in the artificial leg joint implant placement surgery. With this configuration, in artificial leg joint implant placement surgery, the surgeon can more readily measure the positional relationship between the tibia distal part cutting guide 7 and the talus 104 of the patient.

Regarding the measurement instrument 1, the tibia distal part cutting guide 7 is a jig for installing the artificial leg joint implant 2 in the body of the patient 100. With this configuration, in the case of temporarily installing the tibia distal part cutting guide 7 that is used in artificial leg joint implant placement surgery on the tibia 101 of the patient 100, the surgeon can more readily measure the relative positions between the tibia 101 of the patient 100 and the tibia distal part cutting guide 7.

Regarding the measurement instrument 1, the laser application portion 15 is configured to apply the laser beam L1 to the knee joint center 105 of the patient 100, in a state of being supported by the tibia distal part cutting guide 7 via the connecting portion 18. With this configuration, in artificial leg joint implant placement surgery, the surgeon can more readily measure the position of the tibia component 3 to be installed in the distal part 101b of the tibia 101 of the patient and the position of the knee joint center 105. In addition, the laser application portion 15 is installed on the tibia distal part cutting guide 7. With this configuration, the laser application portion 15 is held in a stable orientation by the distal part 101b of the tibia 101. Accordingly, the surgeon can more accurately measure the positions using the laser beam L1 in a state where the position of the laser beam L1 is less likely to shift, unlike in the case of a configuration in which, when the surgeon uses a measurement rod instead of the laser application portion 15 and holds the measurement rod, the position of the measurement rod is likely to shift.

Regarding the measurement instrument 1, the laser application portion 15 is configured to radially apply the laser beam L1 to the patient 100. With this configuration, the laser beam L1 is applied to more portions. Thus, the surgeon can more readily visually check the positional relationship between each portion to which the laser beam L1 is applied and a reference position (the tibia distal part cutting guide 7).

### Second Embodiment

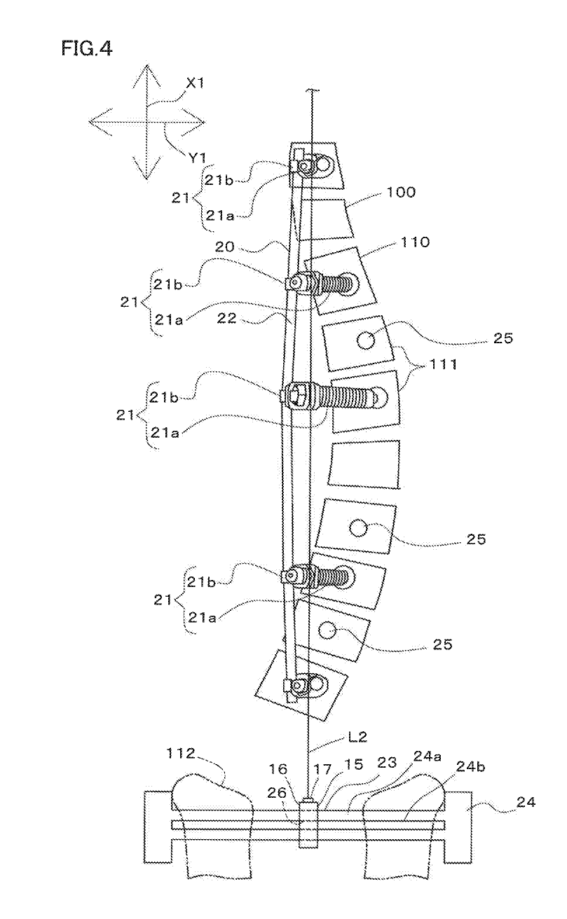

FIG. 4 is a rear view showing a measurement instrument 23 according to the second embodiment of the present invention and a portion of the skeleton of the patient 100. This embodiment will describe the main points regarding spine correction surgery to perform treatment on vertebrae 111 of the patient 100, with reference to FIG. 4. Spine correction surgery is correction surgery to bring the shape of the spine 110 of the patient 100, the spine 110 curving due to scoliosis or the like, close to the original shape of the spine 110.

Note that FIG. 4 only partially shows a portion of the skeleton and the like of the patient 100. However, spine correction surgery is performed with an incision made only in a portion around the spine 110 of the patient 100. A surgical device 20 is used in spine correction surgery.

The surgical device 20 has a plurality of fixing screws 21, a correction rod 22, and a measurement instrument 23.

The fixing screws 21 are each provided as a portion that is fixed to any one of the vertebrae 111 of the spine 110 and is connected to the other fixing screws 21 via the correction rod 22. The number of fixing screws 21 that are to be used in spine correction surgery is appropriately set in accordance with the symptoms of the patient 100, for example.

The fixing screws 21 each has an male screw portion 21a, and a rod holder portion 21b, which is attached to the male screw portion 21a.

The male screw portion 21a is fixed to a corresponding vertebra 111 by being screwed into the vertebra 111 from the back side of the patient 100. The rod holder portion 21b is attached to the male screw portion 21a via a ball joint (not shown), and can pivot around the male screw portion 21a. A through hole, through which the correction rod 22 passes, is formed in the rod holder portion 21b. The correction rod 22, which passes through this through hole, is arranged so as to extend in an up-down direction X1 of the patient 100. The correction rod 22 connects the plurality of fixing screws 21 to one another by passing through the through holes of the plurality of fixing screws 21.

In spine correction surgery, the fixing screws 21, to which the correction rod 22 is attached, are screwed into the corresponding vertebrae 111, and the vertebrae 111 are thus brought toward the correction rod 22. Thus, the shape of the spine 110 is corrected to have a shape extending in the up-down direction X1. Then, the orientation of the spine 110, which has been corrected using the fixing screws 21 and the correction rod 22, is measured using the measurement instrument 23. As a result, whether or not the orientation of the corrected spine 110 is a desired orientation is checked.

Note that, although not shown in the diagram, one more correction rod 22 is connected to the spine 110 using a plurality of fixing screws 21. The two correction rods 22 are connected so as to be parallel to each other, using a connecting member (not shown).

The measurement instrument 23 has a fixed jig 24, the laser application portion 15, a connecting portion 26, and marker members 25.

The fixed jig 24 is provided as a jig that is to be temporarily fixed to the pelvis 112 of the patient 100. The fixed jig 24 is formed in a rectangular frame shape, for example. The fixed jig 24 may be provided in a plurality of sizes according to the physique of the patient 100, or one type of the fixed jig 24 may be configured so that the size of the fixed jig 24 can be changed in accordance with the shape of the pelvis 112 of the patient 100.

The fixed jig 24 is fixed to the patient 100 by sandwiching the pelvis 112 of the patient 100 from both sides in a left-right direction Y1, for example. A front portion 24a of the fixed jig 24 is arranged on the front face side of the patient 100, and extends in the left-right direction Y1. A rail 24b is formed in this front portion 24a. The rail 24b extends straight in the left-right direction Y1. The laser application portion 15 is supported by the rail 24b in the front portion 24a via the connecting portion 26.

In this embodiment, the laser application portion 15 is configured to apply a laser beam L2 to measure the relative positional relationship between the vertebrae 111 of the patient 100.

The laser application portion 15 is installed to the fixed jig 24 so that the laser beam L2 extends in a direction that coincides with the up-down direction X1 of the patient 100 (the direction in which the head and the end of the leg are connected; the direction in which the spine 110 originally extends). The application face 17 of the laser application portion 15 faces toward the head of the patient 100.

The connecting portion 26 is provided in order to connect the laser application portion 15 to the fixed jig 24. For example, the connecting portion 26 is a plate-shaped attachment member, and is fixed to the casing 16 of the laser application portion 15. A groove portion into which the rail 24b fits is formed in the connecting portion 26. The connecting portion 26 can slide relative to the rail 24b in the longitudinal direction of the rail 24b (left-right direction Y1).

The laser application portion 15 is arranged so as to apply the laser beam L2 that extends in the up-down direction X1 of the patient 100, in a state of being supported by the fixed jig 24 via the connecting portion 26. That is to say, the laser application portion 15 applies the laser beam L2 in order to measure the positional relationship between the plurality of vertebrae 111 of the spine 110. The position of the laser beam L2 is set in the left-right direction Y1 so as to coincide with the position of at least one marker member 25.

The marker member 25 is attached to a vertebra 111 to which a fixing screw 21 has been attached, or a vertebra 111 to which no fixing screw 21 has been attached. The marker member 25 is provided as a member serving as a mark during measurement using the laser beam L2 from the laser application portion 15. For example, the marker member 25 is a round shaft member, and is fixed to one of the vertebrae 111 by being driven into the back side of this vertebra 111. The portion at which the marker member 25 is to be installed is set during pre-surgery planning. For example, marker members 25 are installed on a vertebra 111 on one end side of the spine 110, a vertebra 111 located substantially at the center, and a vertebra 111 on the other end side in the up-down direction X1.

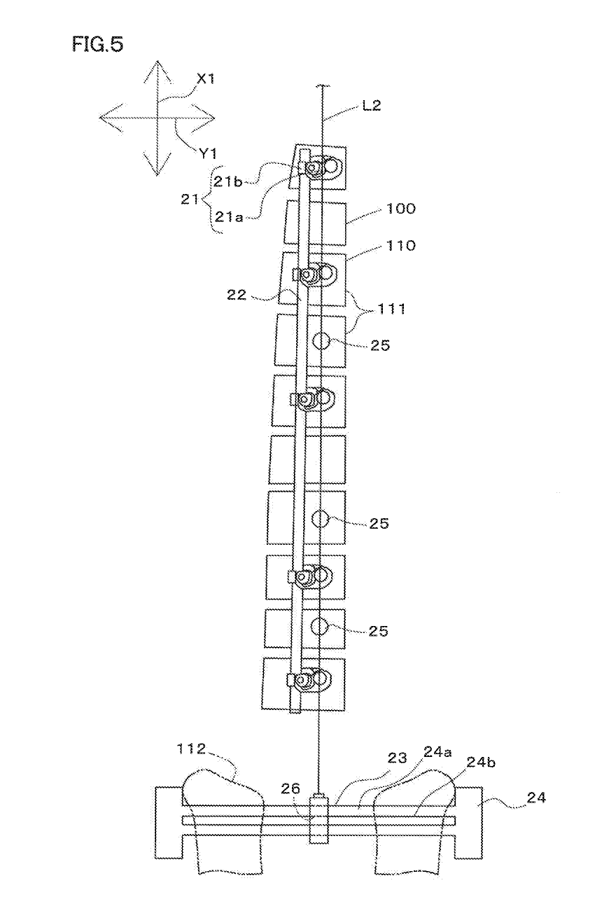

Then, for example, if all marker members 25 are aligned with the laser beam L2 when the back of the patient 100 is seen from the back side as shown in FIG. 5, it indicates that the spine 110 extends substantially straight in a desired direction (up-down direction X1). In this case, it is determined that spine correction surgery has been correctly performed.

On the other hand, if at least some of the marker members 25 are not aligned with the laser beam L2 when the back of the patient 100 is seen from the back side as shown in FIG. 4, it indicates that the spine 110 does not extend straight in the desired direction (up-down direction X1). In this case, the surgeon readjusts the position of the vertebrae 111 so that all marker members 25 are aligned with the laser beam L2 when the patient 100 is seen from the back side. Note that the laser application portion 15 is configured to radially emit the laser beam L2 when seen from the side. Thus, the laser beam L2 is applied to a plurality of portions on the skin surface of the back of the patient 100.

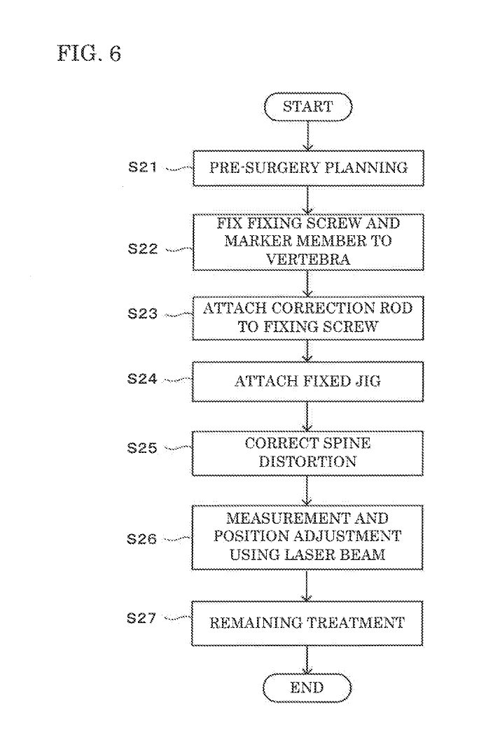

Next, an overview of a procedure for spine correction surgery will be described. FIG. 6 is a flowchart showing the main points of an exemplary flow of spine correction surgery.

Referring to FIG. 6, in spine correction surgery, first, pre-surgery planning is carried out (step S21). In the pre-surgery planning, first, the upper half of the body of the patient 100 that includes an affected area of the patient 100 and the surrounding area of the affected area is subjected to X-ray imaging or CT imaging. The surgeon then determines the number and installation positions of the fixing screws 21 to be used in spine correction surgery, and the number and installation positions of the marker members 25, based on images obtained through X-ray imaging or CT imaging.

Next, the surgeon begins surgery. Specifically, the surgeon installs the fixing screws 21 and marker members 25 on predetermined vertebrae 111 that have been determined during the pre-surgery planning (step S22). At this time, the fixing screws 21 are installed so that the amount of screwing the fixing screws 21 into the corresponding vertebrae 111 is smaller than that at the time when the surgery is complete. Note that the marker members 25 may be installed on the fixing screws 21.

Next, the correction rod 22 is arranged so as to pass through the through holes in the rod holder portions 21b of the fixing screws 21. Thus, the correction rod 22 is attached to the fixing screws 21 (step S23). As a result, the fixing screws 21 are connected to one another via the correction rod 22.

Next, the surgeon attaches the fixed jig 24 to the pelvis 112 of the patient 100 (step S24). Next, the surgeon corrects distortion of the spine 110 (step S25). Specifically, the surgeon displaces the vertebrae 111, to which the fixing screws 21 are fixed, toward the correction rod 22 by appropriately increasing the amount of screwing the fixing screws 21 into the corresponding vertebrae 111.

Thereafter, the surgeon performs a measurement operation and an operation to adjust the position of the spine 110 using the laser beam L2 applied from the laser application portion 15 installed on the fixed jig 24 (step S26). More specifically, the surgeon checks whether or not the laser beam L2 applied in a direction parallel to the up-down direction X1 is aligned with all of the plurality of marker members 25 arranged in the up-down direction X1 when the patient 100 is seen from the back side. If the laser beam L2 is aligned with all of the plurality of marker members 25 arranged in the up-down direction X1 when the patient 100 is seen from the back side, the surgeon determines that the spine 110 has been correctly corrected as per the pre-surgery planning.

On the other hand, if the laser beam L2 is not aligned with at least one of the plurality of marker members 25 arranged in the up-down direction X1 when the patient 100 is seen from the back side, the surgeon determines that the spine 110 has not been correctly corrected in accordance with the pre-surgery planning. In this case, the surgeon appropriately resets the amount by which the respective fixing screws 21 are screwed in. The surgeon thus adjusts the position of the vertebrae 111 relative to the position of the correction rod 22. Thereafter, the surgeon performs the remaining treatment, such as stitching up the incision portion of the patient 100 (step S27).

As described above, in the measurement instrument 23, the laser application portion 15 is configured to apply the laser beam L2 to measure the positional relationship between the vertebrae 111 of the patient 100. With this configuration, the surgeon does not need to hold a heavy item, such as a measurement rod, in order to measure the positional relationship concerning the vertebrae 111 of the patient 100. Accordingly, the burden on the surgeon when measuring the positional relationship concerning the vertebrae 111 of the patient 100 can be reduced. In addition, the laser beam L2 can be formed in a thinner line than a measurement rod. Accordingly, the laser beam L2 can be more readily and accurately applied to the vertebrae 111 of the patient 100 and the marker members 25. As a result, the surgeon can more readily perform a measurement operation using the measurement instrument 23.

In the measurement instrument 23, the laser application portion 15 is configured to apply the laser beam L2 in order to measure the relative positions between the plurality of vertebrae 111 of the patient 100. With this configuration, the relative positions between the plurality of vertebrae 111 can be more readily measured in spine correction surgery to correct the relative positions between the plurality of vertebrae 111 of the patient 100. That is to say, during correction surgery to treat scoliosis, i.e. an unnaturally curved spine 110 of the patient 100, for example, the measurement instrument 23 can be used in order to measure the alignment direction of the plurality of the vertebrae 111.

Regarding the measurement instrument 23, the laser application portion 15 is configured to apply the laser beam L2 in order to measure the plurality of vertebrae 111 of the spine 110, in a state of being supported by the fixed jig 24 via the connecting portion 26. With this configuration, in spine correction surgery, the surgeon can more readily measure the alignment direction of the spine 110, for example. The laser application portion 15 is installed on the fixed jig 24. Thus, the laser application portion 15 is held in a stable orientation by the fixed jig 24. Accordingly, the surgeon can more accurately measure positions using the laser beam L2 in a state where the position of the laser beam L2 is less likely to shift, unlike in the case of a configuration in which, when the surgeon uses a measurement rod instead of the laser application portion 15 and holds the measurement rod, the measurement rod position is likely to shift.

### Third Embodiment

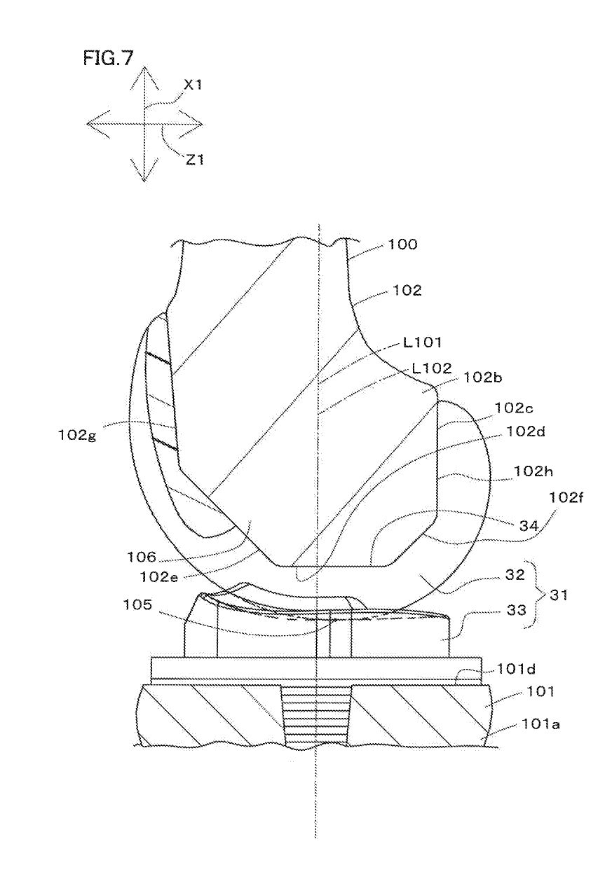

FIG. 7 is a diagram illustrating the third embodiment of the present invention, and is a side view with a partial cross section showing a state where an artificial knee joint implant 31 has been installed in the patient 100. The third embodiment of the present invention will describe the main points of artificial knee joint implant placement surgery, which is implant placement surgery to perform treatment on a bone of a patient, with reference to FIG. 7. Note that, in this embodiment, uneven shapes of the surfaces of the tibia 101 and femur 102 are schematically shown with mesh lines.

Artificial knee joint implant placement surgery is surgery to install the artificial knee joint implant 31 in a knee joint 106, which includes the distal part 102b of the femur 102 and the proximal part 101a of the tibia 101 of the patient 100. Note that, although FIG. 7 shows the skeleton of the patient 100, artificial knee joint implant placement surgery is performed with an incision made only in the periphery of the knee joint 106 of the patient 100.

The artificial knee joint implant 31 has a femur component 32, which is to be fixed to the distal part 102b of the femur 102 of the patient 100, and a tibia component 33, which is to be fixed to the proximal part 10a of the tibia 101.

A portion of the femur component 32 that is to be received by the tibia component 33 is formed in a protruding curved shape. A portion of the tibia component 33 that is to be received by the femur component 32 is formed in a recessed shape. The femur component 32 and the tibia component 33 relatively slide against each other with bending motion of the knee of the patient 100. Thus, the bending motion of the tibia 101 relative to the femur 102 is guided through the cooperation of the femur component 32 and the tibia component 33.

A fixed face 34 is formed on an inner face of the femur component 32 that faces the distal part 102b side of the femur 102. The fixed face 34 is provided in order to fix the femur component 32 to a cut-bone face 102c of the femur 102.

The cut-bone face 102c is a face that is artificially formed by the surgeon in artificial knee joint implant placement surgery. The cut-bone face 102c is formed as a result of the surgeon making an incision in a portion of the distal part 102b of the femur 102 using an instrument such as a cutter.

The cut-bone face 102c has a main face 102d, which is arranged substantially horizontally when the patient 100 assumes an upright posture on a horizontal surface, a pair of inclined faces 102e and 102f, which extend from the front end and rear end, respectively, of the main face 102d, and a pair of opposing faces 102g and 102h, which are arranged on the front end side and back end side, respectively, of the femur 102 and extend from the pair of inclined faces 102e and 102f toward the proximal part of the femur 102.

A procedure for forming the main face 102d, the pair of inclined faces 102e and 102f, and the pair of opposing faces 102g and 102h will be described later in detail.

The tibia component 33 is fixed to a cut-bone face 101d, which is formed in the proximal part 101a of the tibia 101. The cut-bone face 101d is a face that is artificially formed by the surgeon in artificial knee joint implant placement surgery. The cut-bone face 101d is formed as a result of the surgeon making an incision in a leading end face of the proximal part 101a using an instrument such as a cutter, for example. The cut-bone face 101d is formed so as to extend substantially horizontally when the patient 100 assumes an upright posture on a horizontal surface, for example.

Next, a description will be given of the main points of a procedure for forming the cut-bone face 101d of the tibia 101, and the main points of the procedure for forming the cut-bone face 102c of the femur 102.

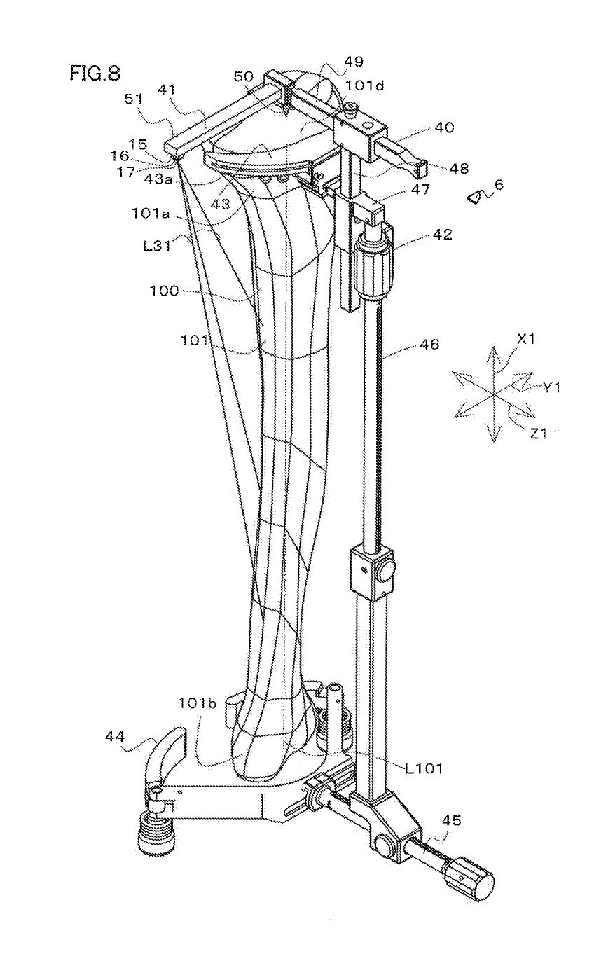

First, the main points of the procedure for forming the cut-bone face 101d of the tibia 101 will be described. FIG. 8 is a perspective view showing a state where a surgical device 40 has been attached to the tibia 101. Referring to FIG. 8, the surgical device 40 is provided in order to guide displacement of the cutter when forming the cut-bone face 101d in the proximal part 101a of the tibia 101.

The surgical device 40 has a measurement instrument 41, a guide member holder portion 42, and a guide member 43.

The guide member holder portion 42 is a jig for installing the artificial knee joint implant 31 in the body of the patient 100, and is an example of an “instrument to be used in implant placement surgery” according to the present invention.

The guide member holder portion 42 has a clamp 44, a shaft 45, a first rod 46, an attachment 47, a second rod 48, a spike rod 49, and a spike 50.

The clamp 44 is an example of a “distal side portion to be connected to a distal part of a tibia” according to the present invention. The clamp 44 is a member that is to be fixed to the patient 100 as a result of clamping the leg of the patient 100 around the distal part 101b of the tibia 101 of the patient 100, and is formed in a substantially C-shape. The shaft 45 extends from the clamp 44 in a front-rear direction Z1. The position of the shaft 45 in the left-right direction Y1 is arranged so as to be aligned with the position of the axis of the tibia 101. The first rod 46 is attached to the shaft 45.

The first rod 46 is a member that extends in the up-down direction X1, and is configured to extend and contract. Note that the first rod 46, the second rod 48, and the spike rod 49 are examples of a “rod to connect a proximal side portion and a distal side portion to each other” according to the present invention. One end of the first rod 46 is connected to the shaft 45, and is configured so that its position can be adjusted in the front-rear direction Z1 relative to this shaft 45. The other end of the first rod 46 supports the attachment 47.

The attachment 47 supports the second rod 48 so that the second rod 48 can be displaced in the up-down direction X1, and supports the guide member 43. A through hole that extends in the up-down direction X1 is formed in the attachment 47, and the second rod 48 passes through this through hole. The spike rod 49 is attached to the second rod 48. The spike rod 49 is a shaft member that extends in the front-rear direction Z1, and is configured so that its position can be adjusted in the front-rear direction Z1 relative to the second rod 48. The spike rod 49 is arranged adjacent to the proximal part 101a of the tibia 101. The spike 50 is fixed to one end of the spike rod 49.

The spike 50 is an example of a “proximal side portion to be connected to a proximal part of a tibia” according to the present invention. The spike 50 is a protruding member and is provisionally fixed (temporarily fixed) to the tibia 101 as a result of being driven into an end face of the proximal part 101a of the tibia 101. With this configuration, the clamp 44 is supported at the distal part 101b of the tibia 101 at one end of the guide member holder portion 42. The spike 50 is supported at the proximal part 101a of the tibia 101 at the other end of the guide member holder portion 42. The guide member holder portion 42 is thus installed on the patient 100. As mentioned above, the guide member 43 is installed on the guide member holder portion 42.

The guide member 43 is provided for guiding a bone-cutting position when the surgeon cuts the bone in the proximal part 101a of the tibia 101. The guide member 43 is supported by the attachment 47. The guide member 43 is a member that extends in an elongated manner in the left-right direction Y1. A slit hole 43a, which extends in the left-right direction Y1, is formed in the guide member 43. The slit hole 43a faces the proximal part 101a side of the tibia 101. The surgeon inserts the cutter 6 into this slit hole 43a, and thus performs bone-cutting treatment on the proximal part 101a of the tibia 101 using the cutter 6 in a state of being guided by the guide member 43. The position, orientation, and the like of the guide member 43 are measured by the measurement instrument 41.

The measurement instrument 41 has the laser application portion 15 and a connecting portion 51.

In this embodiment, the laser application portion 15 is configured to apply a laser beam L31 in order to measure the positional relationship between an axis L101 of the tibia 101 and the guide member 43 (guide member holder portion 42) that is used in artificial knee joint implant placement surgery.

In this embodiment, the laser application portion 15 is installed on the guide member holder portion 42 so that the laser beam L31 is parallel to the axis L101 of the tibia 101 when seen from the side. The laser application portion 15 is supported, via the connecting portion 51, by the spike rod 49 of the guide member holder portion 42, and the application face 17 of the laser application portion 15 faces the distal part 101b side of the tibia 101 of the patient 100.

The connecting portion 51 is provided in order to connect the laser application portion 15 to the spike rod 49 of the guide member holder portion 42. The connecting portion 51 is a rod-shaped attachment member that extends straight, for example. One end of the connecting portion 51 is fixed to the spike rod 49. The other end of the connecting portion 51 is arranged on a side of the proximal part 10a of the tibia 101 in the left-right direction Y1, and fixes the casing 16 of the laser application portion 15.

The laser application portion 15 is configured to apply the laser beam L31 to measure the parallelism between the first rod 46 and the axis L101 of the tibia 101, in a state of being supported by the guide member holder portion 42 via the connecting portion 51. Note that the laser application portion 15 is configured to radially emit the laser beam L31. Thus, the laser beam L31, which extends in the up-down direction X1 when seen from the side, is applied to a plurality of portions on the skin surface of the leg of the patient 100.

In this case, the surgeon adjusts, for example, the position of the first rod 46 in the front-rear direction Z1 relative to the shaft 45. Thus, the inclination angles of the first rod 46 and the guide member 43 relative to the axis L101 of the tibia 101 are adjusted, with the spike 50 acting as a fulcrum. As a result, the positions of the first rod 46, the guide member 43, and the like are adjusted so that the first rod 46 is parallel to the axis L101 of the tibia 101.

When the first rod 46 of the guide member holder portion 42 is parallel to the axis L101 of the tibia 101, i.e. when the orientation of the slit hole 43a in the guide member 43 is substantially perpendicular to the axis L101 of the tibia 101, the positioning of the guide member holder portion 42 (guide member 43) is complete.

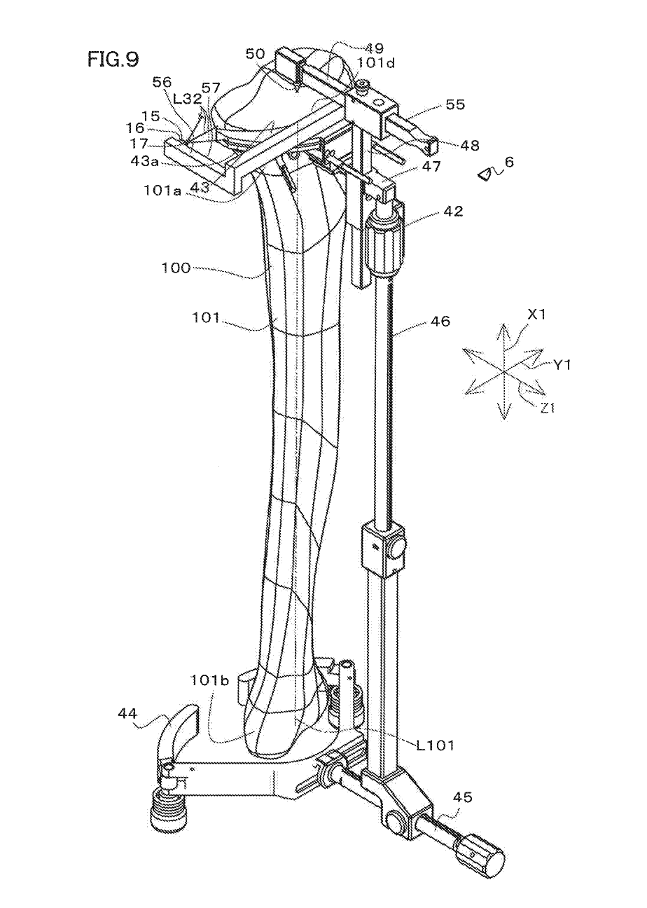

Next, the surgeon uses a measurement instrument 56 in order to check the position of the slit hole 43a of the guide member 43. FIG. 9 is a perspective view showing a surgical device 55 and the like. Referring to FIG. 9, the surgical device 55 has the measurement instrument 56, the guide member holder portion 42, and the guide member 43.

The measurement instrument 56 has the laser application portion 15 and a connecting portion 57.

In the measurement instrument 56, the laser application portion 15 is configured to apply a laser beam in order to measure the positional relationship between the axis L101 of the tibia 101 and the guide member 43 (guide member holder portion 42) that is used in artificial knee joint implant placement surgery.

In this embodiment, the laser application portion 15 is installed on the guide member holder portion 42 so that the laser beam L32 extends in a plane that is substantially perpendicular to the axis L101 of the tibia 101. The laser application portion 15 is supported by the spike rod 49 of the guide member holder portion 42 via the connecting portion 57, and the application face 17 of the laser application portion 15 faces the proximal part 101a side of the tibia 101 of the patient 100.

The connecting portion 57 is provided in order to connect the laser application portion 15 to the spike rod 49 of the guide member holder portion 42. The connecting portion 57 is an L-shaped attachment member, for example. One end of the connecting portion 57 is fixed to the spike rod 49. The other end of the connecting portion 57 is arranged on a side of the proximal part 101a of the tibia 101 in the left-right direction Y1, and fixes the casing 16 of the laser application portion 15.

The laser application portion 15 is configured to apply the laser beam L32 in order to measure the bone-cutting position in the proximal part 101a of the tibia 101, in a state of being supported by the guide member holder portion 42 via the connecting portion 57. Note that the laser application portion 15 is configured to radially emit the laser beam L32 when the surgeon looks at the patient 100 along the up-down direction X1. With this configuration, the laser beam L32, which extends in the front-rear direction Z1 when seen from the side, is applied to a plurality of portions on the skin surface of the leg of the patient 100.

In this case, the surgeon adjusts the guide member 43 in the up-down direction X1 relative to the first rod 46, for example. Thus, the position of the guide member 43 in the up-down direction X1 (a direction parallel to the axis L101 of the tibia 101) is adjusted. As a result, the bone-cutting position in the proximal part 101a of the tibia 101 in the up-down direction X1 is set.

The surgeon inserts the cutter 6 into the slit hole 43a of the guide member 43, whose position has been determined, and forms the cut-bone face 101d, which is substantially perpendicular to the axis L101, in the proximal part 101a of the tibia 101 of the patient 100.

Next, the surgeon performs an operation to form the cut-bone face 102c in the distal part 102b of the femur 102. A description will be given below of the main points of a procedure by which the surgeon forms the cut-bone face 102c of the femur 102.

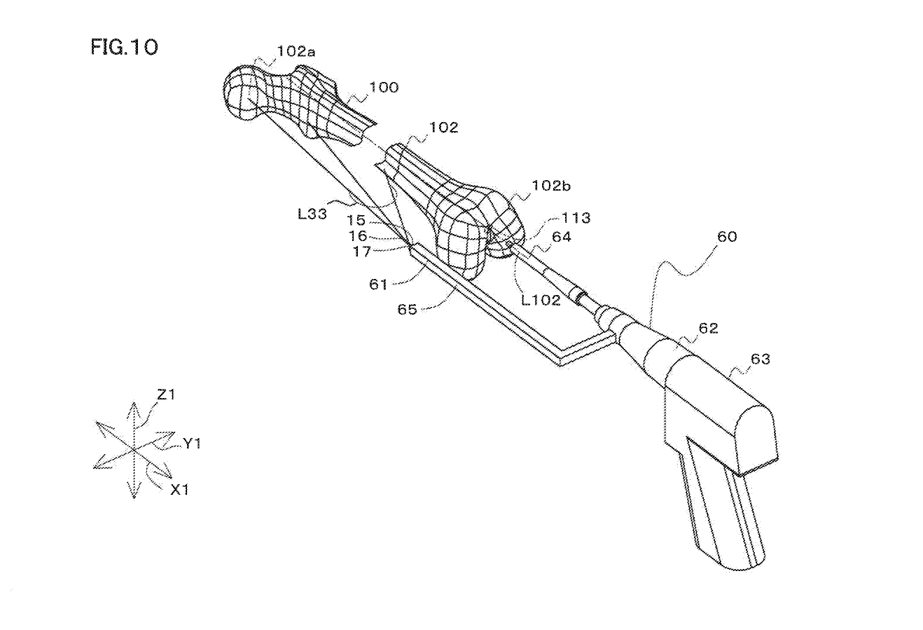

FIG. 10 is a perspective view showing the main points to illustrate a procedure for forming a reamer hole 113 in the distal part 102b of the femur 102. Referring to FIG. 10, when forming the cut-bone face 102c in the femur 102, the surgeon first forms the reamer hole 113 in the distal part 102b of the femur 102. The reamer hole 113 is formed using a surgical device 60.

The surgical device 60 has a measurement instrument 61 and a drill 62.

The drill 62 is a jig for installing the artificial knee joint implant 31 in the body of the patient 100, and is an example of an “instrument to be used in implant placement surgery” according to the present invention. The drill 62 is an electric drill for forming the reamer hole 113 in the distal part 102b of the femur 102.

The drill 62 has a casing 63 and a drill body 64.

The casing 63 is a portion to be held by the surgeon. The casing 63 has a grip portion to be gripped by the surgeon, and a housing portion to house an electric motor and a battery to drive this electric motor. The drill body 64 extends from the housing portion.

The drill body 64 is a shaft-shaped member having a blade portion, and rotates with the rotation of an output shaft of the electric motor arranged in the casing 16. The distal part 102b of the femur 102 is shaved due to this rotation of the drill body 64, and the reamer hole 113 is thus formed.

The orientation of the drill body 64 relative to the femur 102 is measured by the measurement instrument 61. The measurement instrument 61 has the laser application portion 15 and a connecting portion 65.

In the measurement instrument 61, the laser application portion 15 is configured to apply a laser beam L33 in order to measure the positional relationship between the femur 102 and the drill body 64 of the drill 62 that is used in artificial knee joint implant placement surgery.

In this embodiment, the laser application portion 15 is installed on the casing 63 of the drill 62 so that the laser beam L33 is substantially aligned with an axis L102 of the femur 102 when seen from the side. The laser application portion 15 is supported by the casing 63 of the drill 62 via the connecting portion 65, and the application face 17 of the laser application portion 15 faces toward the distal part 102b of the femur 102 side.

The connecting portion 65 is provided in order to connect the laser application portion 15 to the casing 63 of the drill 62. The connecting portion 65 is a portion that is to be connected to the drill 62 when the reamer hole 113 is formed in the distal part 102b of the femur 102. The connecting portion 65 is an L-shaped attachment member, for example.

One end of the connecting portion 65 is fixed near a portion of the casing 63 at which the drill body 64 protrudes. The other end of the connecting portion 65 is arranged on a side of the distal part 102b of the femur 102 in the left-right direction Y1, and fixes the casing 16 of the laser application portion 15.

The laser application portion 15 is configured to apply a laser beam L33 in order to measure the coaxiality between the drill body 64 of the drill 62 and the axis L102 of the femur 102, in a state of being supported by the casing 63 of the drill 62 via the connecting portion 65. The laser application portion 15 is configured to radially emit the laser beam L33 when the patient 100 is seen from the side. Thus, the laser beam L33, which extends in the up-down direction X1, is applied to a plurality of portions on the skin surface on a side face of the leg of the patient 100.

In this case, the surgeon adjusts the position of the drill 62 so that the axis of the drill body 64 substantially coincides with the axis L102 of the femur 102. The surgeon forms the reamer hole 113 in the distal part 102b of the femur 102 using the drill 62, in a state where the axis of the drill body 64 substantially coincides with the axis L102 of the femur 102.

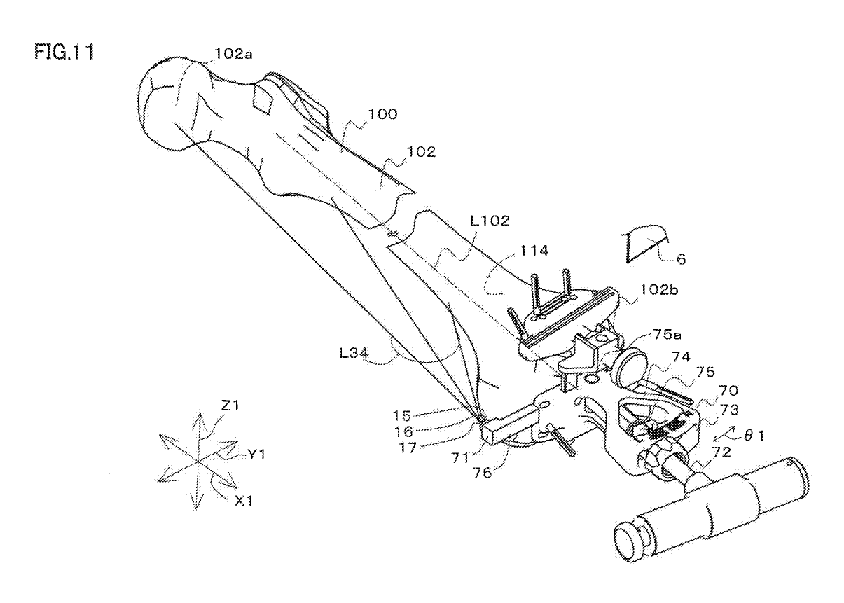

FIG. 11 is a perspective view showing the main points to illustrate a procedure for inserting a medullary cavity rod 72 into the distal part 102b of the femur 102. Referring to FIG. 11, the surgeon forms the reamer hole 113 in the femur 102, and thereafter inserts the medullary cavity rod 72 into the femur 102. The medullary cavity rod 72 is a portion of a surgical device 70.

The surgical device 70 has a measurement instrument 71, the medullary cavity rod 72, and a valgus alignment guide 73.

The medullary cavity rod 72 is a rod-shaped member that extends straight and is to be inserted into a medullary cavity portion 114 of the femur 102 of the patient through the reamer hole 113 (not shown in FIG. 11). The medullary cavity rod 72 is also called an IM (Intra Medullary rod), and is used to indicate the axis L102 of the femur 102. The medullary cavity rod 72 is inserted into the femur 102 so as to be coaxially aligned with the femur 102, by the valgus alignment guide 3.

The medullary cavity rod 72 and the valgus alignment guide 73 are jigs to install the artificial knee joint implant 31 in the body of the patient 100, and are examples of the “instrument to be used in implant placement surgery” according to the present invention and are also examples of the “jig”. The valgus alignment guide 73 is attached to the femur 102 in order to guide the insertion of the medullary cavity rod 72 into the medullary cavity portion 114 of the femur 102, and the position of the medullary cavity rod 72 can be adjusted in a valgus angle direction θ1 of the femur 102.

The valgus alignment guide 73 is installed in the distal part 102b of the femur 102. The valgus angle direction θ1 refers to a direction moving around an intersection point between an axis that passes through the femur 102 and is parallel to the up-down direction X1 and the axis of the femur 102 when seen from the front.

The valgus alignment guide 73 has a body member 74 and a pivot member 75.

The body member 74 is fixed to the distal part 102b of the femur 102 using a pin or the like (not shown). The body member 74 is formed in a substantially T-shape. The pivot member 75 is provided as a portion that pivotally supports the medullary cavity rod 72.

The pivot member 75 is formed in an elongated cylindrical shape. A shaft portion 75a of the pivot member 75 is pivotably connected to the body member 74. The medullary cavity rod 72 is inserted in the pivot member 75. The pivot member 75 can pivot around the shaft portion 75a together with the medullary cavity rod 72.

The orientation of the medullary cavity rod 72 relative to the femur 102 is measured by a measurement instrument 71. The measurement instrument 71 has the laser application portion 15 and a connecting portion 76.

In the measurement instrument 71, the laser application portion 15 is configured to apply a laser beam L34 in order to measure the positional relationship between the femur 102, and the medullary cavity rod 72 and valgus alignment guide 73 that are used in artificial knee joint implant placement surgery.

In the measurement instrument 71, the laser application portion 15 is installed on the body member 74 of the valgus alignment guide 73 so that the laser beam L34 is substantially aligned with the axis L102 of the femur 102 when seen from the side. The laser application portion 15 is supported by the body member 74 via the connecting portion 76, and the application face 17 of the laser application portion 15 faces a bone head center 102a side of the femur 102 of the patient 100.

The connecting portion 76 is an L-shaped attachment member, for example. One end of the connecting portion 76 is fixed to one end of the body member 74 in the left-right direction Y1. The other end of the connecting portion 76 is arranged on a side of the distal part 102b of the femur 102 in the left-right direction Y1, and fixes the casing 16 of the laser application portion 15.

The laser application portion 15 is configured to apply a laser beam L34 in order to measure the positional relationship between the axis L102 of the femur 102, and the medullary cavity rod 72 and valgus alignment guide 73, in a state of being supported by the body member 74 of the valgus alignment guide 73 via the connecting portion 76. Note that the laser application portion 15 is configured to radially emit the laser beam L34 when seen from the front. Thus, the laser beam L34, which extends in the up-down direction X1, is applied to a plurality of portions on the skin surface on a side face of the leg of the patient 100.

In this case, the surgeon adjusts the position of the medullary cavity rod 72 so that the laser beam L34 and the medullary cavity rod 72 are aligned with the axis L102 of the femur 102 when seen from the side. Note that, in the surgical device 70, the medullary cavity rod 72 may be omitted.

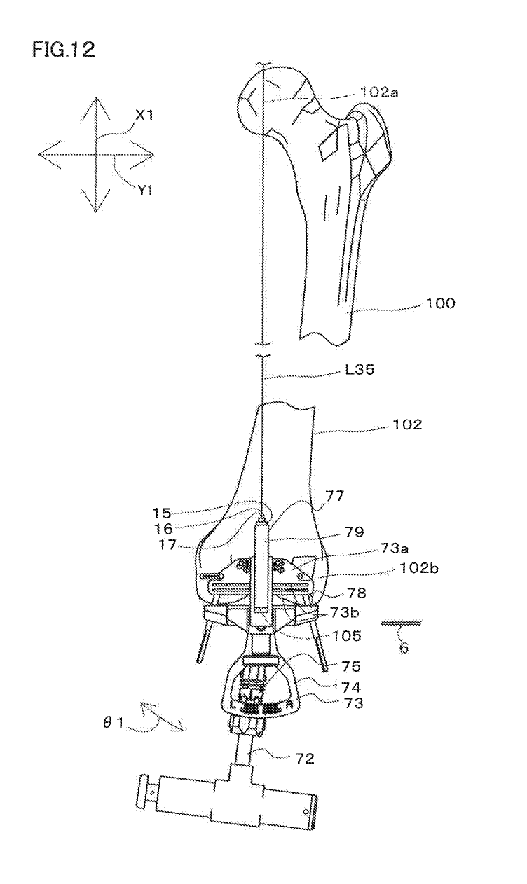

Next, the surgeon uses a measurement instrument 77 in order to check the orientation of the medullary cavity rod 72 in the valgus angle direction θ1. FIG. 12 is a front elevational view showing a surgical device 78 and the like. Referring to FIG. 12, the surgical device 78 has the measurement instrument 77, the medullary cavity rod 72, and the valgus alignment guide 73.

The measurement instrument 77 has the laser application portion 15 and a connecting portion 79.

In the measurement instrument 77, the laser application portion 15 is configured to apply a laser beam L35 in order to measure the positional relationship between a reference axis L100, which passes through the bone head center 102a of the femur 102 and the knee joint center 105 when seen from the front, and the medullary cavity rod 72 that is used in artificial knee joint implant placement surgery.

In this embodiment, the laser application portion 15 is installed on the body member 74 of the valgus alignment guide 73 so that the laser beam L35 extends toward the bone head center 102a of the patient when seen from the front. The laser application portion 15 is supported by the body member 74 via the connecting portion 79, and the application face 17 of the laser application portion 15 faces the bone head center 102a side of the patient 100.

The connecting portion 79 is an L-shaped attachment member, for example. One end of the connecting portion 79 is fixed to the body member 74. The other end of the connecting portion 79 opposes the distal part 102b of the femur 102 in the front-rear direction Z1 (a direction perpendicular to the page of FIG. 12). The casing 16 of the laser application portion 15 is fixed to the other end of the connecting portion 79.

The laser application portion 15 is configured to apply the laser beam L35 in order to indicate the bone head center 102a of the femur 102, in a state of being supported by the body member 74 of the valgus alignment guide 73 via the connecting portion 79. Note that the laser application portion 15 is configured to radially emit the laser beam when seen from the side. Thus, the laser beam L35, which extends in the front-rear direction Z1, is applied to a plurality of portions on the skin surface of the leg of the patient 100.

In this case, for example, the surgeon measures the valgus angle, i.e. the angle formed between the laser beam L35 and the medullary cavity rod 72 when the patient 100 is seen from the front.

Next, the surgeon fixes the guide member 73a to the distal part 102b of the femur 102, with the guide member 73a attached to the valgus alignment guide 73. Thereafter, the surgeon removes the valgus alignment guide 73 and the medullary cavity rod 72 from the femur 102. The surgeon then cuts the bone at the distal part 102b using the cutter 6, in a state where the cutter 6 has been passed through a slit hole 73b in the guide member 73a that is fixed to the distal part 102b of the femur 102. Thus, the main face 102d of the cut-bone face 102c is formed in the distal part 102b of the femur 102.

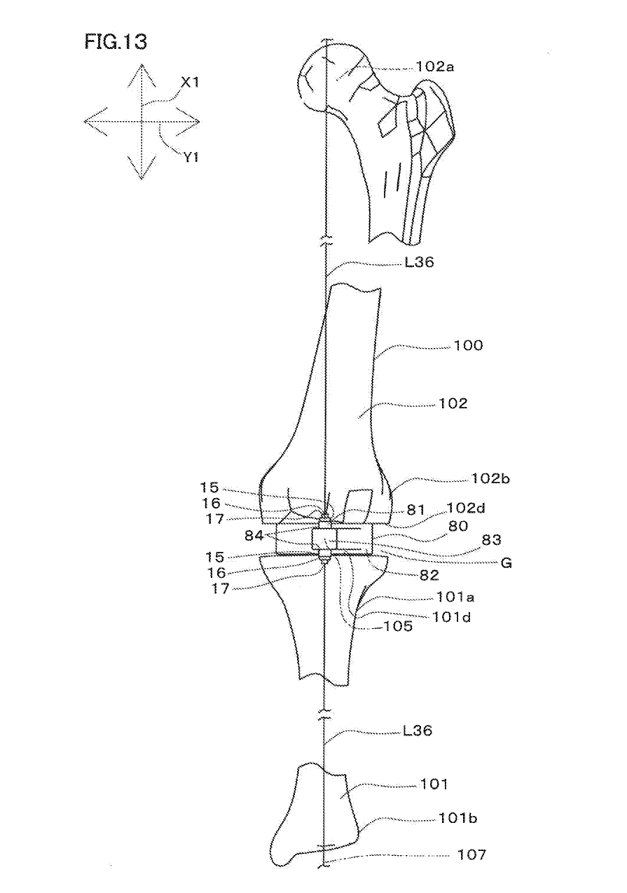

FIG. 13 is a front elevational view illustrating a procedure for checking a gap G between the main face 102d of the cut-bone face 102c of the femur 102 and the cut-bone face 101d of the tibia 101. Referring to FIG. 13, the surgeon forms the main face 102d of the cut-bone face 102c on the femur 102, and thereafter measures the gap G between the cut-bone face 101d of the tibia 101 and the main face 102d. This gap G is measured using the surgical device 80.

The surgical device 80 has a measurement instrument 81 and a spacer 82.

The spacer 82 is a jig for installing the artificial knee joint implant 31 in the body of the patient 100, and is an example of an “instrument to be used in implant placement surgery” according to the present invention. The spacer 82 is a plate-shaped member having a predetermined thickness.

The surgeon selects a spacer 82 that fits the gap G, which has been determined through pre-surgery planning, from among a plurality of spacers 82, which are prepared in advance and have different thicknesses. The spacer 82 is arranged between the main face 102d of the cut-bone face 102c of the femur 102 and the cut-bone face 10d of the tibia 101. An extension portion 83, which has a protruding shape extending from the spacer 82, is provided in a peripheral portion of the spacer 82.

When the surgeon measures the gap G using the spacer 82, the bone head center 102a of the patient 100, the knee joint center 105, and a leg joint center 107 need to be aligned with one another in a straight line when seen from the front. This positional relationship is measured by the measurement instrument 81. The measurement instrument 81 has two laser application portions 15 and a connecting portion 84.

In the measurement instrument 81, the laser application portions 15 are configured to apply laser beams L36 in order to measure the positional relationship between the femur 102, the tibia 101, and the spacer 82 that is used in artificial knee joint implant placement surgery. In this embodiment, the surgeon checks the alignment (arrangement of the bone head center 102a, the knee joint center 105, and the leg joint center 107) and the gap G, using the laser beams L36.

In this embodiment, the application faces 17 of the two laser application portions 15 are arranged in opposite orientations, and apply the laser beams L36 that extend in the up-down direction X1 when seen from the front. In this embodiment, the laser application portions 15 are installed in the extension portion 83 of the spacer 82 so that the laser beams L36 pass through the bone head center 102a of the femur 102 and the leg joint center 107 when seen from the front. The laser application portions 15 are supported by the extension portion 83 of the spacer 82 via the connecting portion 84, which has a block shape.

The two laser application portions 15 are configured to apply the laser beams L36 in order to indicate the bone head center 102a and the leg joint center 107, in a state of being supported by the extension portion 83 of the spacer 82 via the connecting portion 84. Note that the laser application portions 15 are configured to radially emit the laser beams L36 when seen from the side. Thus, the laser beams L36, which extend along the up-down direction X1 side, are applied to a plurality of portions on the skin surface on a front face of the leg of the patient 100.

In this case, the surgeon adjusts the position of the spacer 82 so that the bone head center 102a, the knee joint center 105, and the leg joint center 107 are arranged substantially in a straight line when seen from the front. The surgeon checks the gap G in this state.

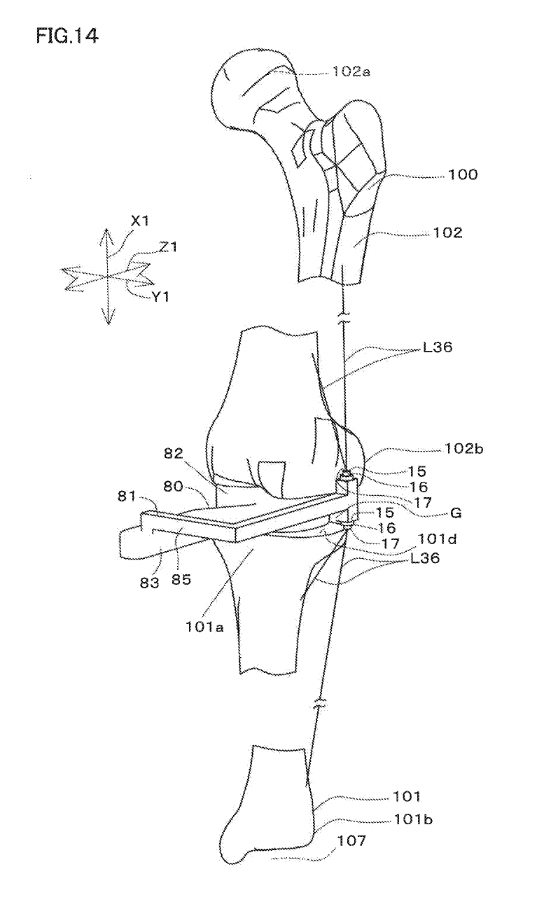

Note that an L-shaped connecting portion 85 may be used instead of the connecting portion 84, as shown in FIG. 14. In this case, one end of the connecting portion 85 is fixed to the extension portion 83. The other end of the connecting portion 85 is arranged on a side of the spacer 82 in the left-right direction Y1. The two laser application portions 15, 15 held at the other end of the connecting portion 85 are configured to apply the laser beams L36 to indicate the bone head center 102a and the leg joint center 107. Note that, in this case, the laser application portions 15 are configured to radially emit the laser beams L36 when seen from the front. Thus, the laser beams L36, which extend in the up-down direction X1, are applied to a plurality of portions on the skin surface on a side face of the leg of the patient 100.

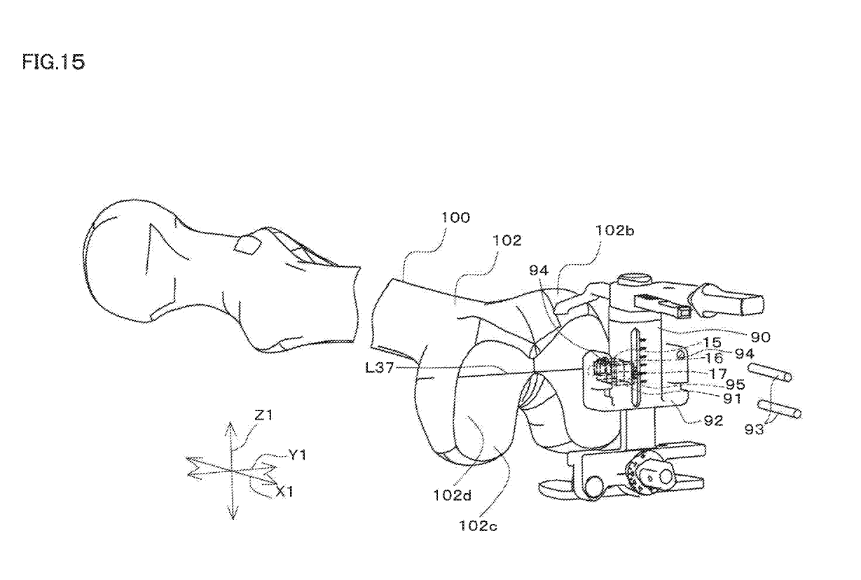

After checking the gap G, the surgeon fixes a sizer member 92, which is shown in FIG. 15, to the distal part 102b of the femur 102.

FIG. 15 is a perspective view showing the main points to illustrate a procedure for fixing the sizer member 92 to the distal part 102b of the femur 102. Referring to FIG. 15, the sizer member 92 is a portion of a surgical device 90.

The surgical device 90 has a measurement instrument 91 and the sizer member 92.

The sizer member 92 is a member that is to be installed in the distal part 102b of the femur 102 of the patient, via pin members 93. The sizer member 92 is a jig for installing the artificial knee joint implant 31 in the body of the patient 100, and is an example of the “instrument to be used in implant placement surgery” according to the present invention and is also an example of the “jig”.

The sizer member 92 is formed as a member that extends in the front-rear direction Z1, in a state of being fixed to the main face 102d of the cut-bone face 102c in the distal part 102b of the femur 102. A pair of pin holes 94 are formed in the sizer member 92. The pin holes 94 are formed as holes into which the pin members 93 are inserted. These pin members 93 are driven into the main face 102d of the cut-bone face 102c in the distal part 102b, in a state of having been inserted in the pin holes 94. That is to say, the sizer member 92 is used to position the pin members 93.

The position of the sizer member 92 relative to the femur 102 is measured by the measurement instrument 91. The measurement instrument 91 has the laser application portion 15 and a connecting portion 95.

In the measurement instrument 91, the laser application portion 15 is configured to apply a laser beam L37 in order to measure the positional relationship between the femur 102 and the sizer member 92 that is used in artificial knee joint implant placement surgery.

In the measurement instrument 91, for example, the laser application portion 15 is installed in the sizer member 92 so that the laser beam L37, which illuminates a projection plane (the main face 102d) in a cross shape, strikes the main face 102d. The laser application portion 15 is supported by the sizer member 92 via the connecting portion 95, which has a block shape, and the application face 17 of the laser application portion 15 faces the main face 102d of the femur 102 of the patient 100.

The laser application portion 15 is configured to apply the laser beam L37 having a cross shape in order to measure the positional relationship between the main face 102d of the cut-bone face 102c and the sizer member 92, in a state of being supported by the sizer member 92 via the connecting portion 95.

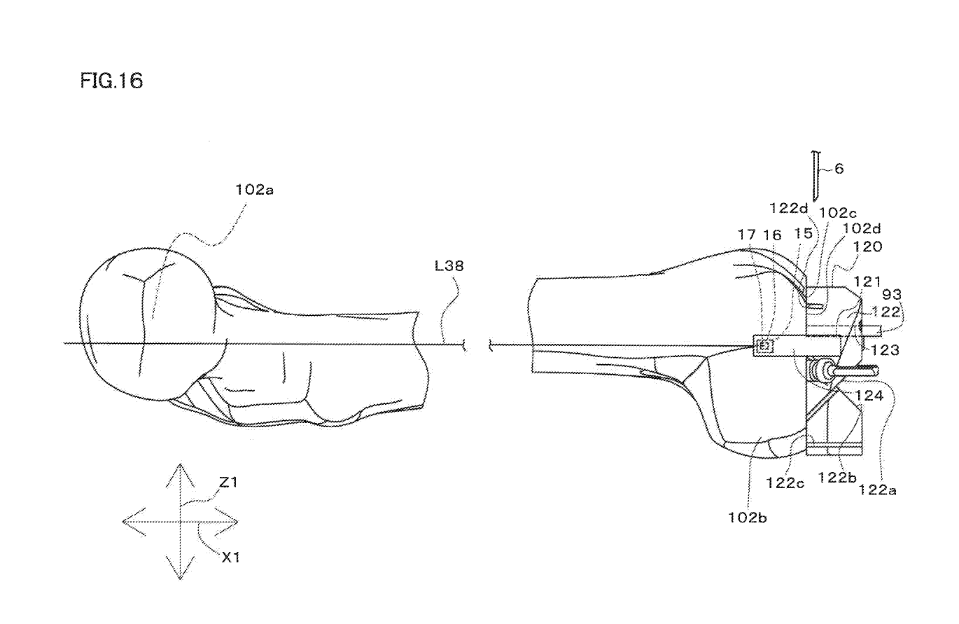

In this case, the surgeon adjusts the position of the sizer member 92 relative to the main face 102d of the cut-bone face 102c, using the laser beam L37 as a mark. After completing the positioning of the sizer member 92 relative to the main face 102d, the surgeon inserts the pin members 93 into the pin holes 94 of the sizer member 92, and fixes these pin members 93 to the femur 102. Next, the surgeon removes the sizer member 92 from the pin members 93. Thereafter, the surgeon attaches a guide member 122, which is shown in FIG. 16, to the pin members 93 (the main face 102d of the cut-bone face 102c of the femur 102).

FIG. 16 is a side view showing the main points to illustrate a procedure for installing the guide member 122 in the distal part 102b of the femur 102. Referring to FIG. 16, the guide member 122 is a member to guide the cutter 6 in order to further form, in the distal part 102b of the femur 102, the faces in the cut-bone face 102c other than the main face 102d, i.e. the pair of inclined faces 102e and 102f and the pair of opposing faces 102g and 102h. The guide member 122 is a portion of a surgical device 120.

The surgical device 120 has a measurement instrument 121 and the guide member 122.

The guide member 122 is a jig for installing the artificial knee joint implant 31 in the body of the patient 100, and is an example of the “instrument to be used in implant placement surgery” according to the present invention and is also an example of the “jig”.

The guide member 122 is a plate-shaped member. A pair of pin holes 123 (one of the pin holes 123 is not shown in FIG. 16) are formed in the guide member 122. Pin members 93 are inserted in the respective pin holes 123, and the guide member 122 is thus supported at the distal part 102b of the femur 102 via the pin members 93. A plurality of slit holes 122a, 122b, 122c, and 122d are formed in the guide member 122.

The slit holes 122a, 122b, 122c, and 122d are formed so as to pass through the guide member 122. The slit holes 122a, 122b, 122c, and 122d are provided as portions that guide displacement of the cutter 6 when the pair of inclined faces 102e and 102f and the pair of opposing faces 102g and 102h of the cut-bone face 102c are formed, respectively.

The orientation of the guide member 122 relative to the femur 102 is measured by a measurement instrument 121. The measurement instrument 121 has the laser application portion 15 and a connecting portion 124.

In the measurement instrument 121, the laser application portion 15 is configured to apply a laser beam L38 in order to measure the positional relationship between the femur 102 and the guide member 122 that is used in artificial knee joint implant placement surgery.

In the measurement instrument 121, for example, the laser application portion 15 is installed on the guide member 122 so that the laser beam L38 is applied to a side face of the leg of the patient 100. The laser application portion 15 is supported by the guide member 122 via the connecting portion 124, and the application face 17 of this laser application portion 15 faces the side face of the leg of the patient 100. The connecting portion 124 is an L-shaped member, for example, and holds the casing 16 of the laser application portion 15.

The laser application portion 15 is configured to apply a laser beam L38 in order to measure the positional relationship between the guide member 122 and the femur 102, in a state of being supported by the guide member 122 via the connecting portion 124.

In this case, the laser beam L38 extends in the up-down direction X1. The laser application portion 15 is configured to radially emit the laser beams L38 when seen from the front. Thus, the laser beam L38, which extends along the up-down direction X1 side, is applied to a plurality of portions on the skin surface on the side face of the leg of the patient 100. The surgeon adjusts the orientation of the guide member 122 relative to the main face 102d of the cut-bone face 102c, using the laser beam L38 as a mark. Next, the surgeon sequentially inserts the cutter 6 into the slit holes 122a, 122b, 122c, and 122d of the guide member 43. As a result, the pair of opposing faces 102g and 102h and the pair of inclined faces 102e and 102f are formed in the distal part 102b of the femur 102. Thereafter, the surgeon removes the pin members 93 and the guide member 122 from the distal part 102b of the femur 102. Thus, the operation to form the cut-bone face 102c in the distal part 102b of the femur 102 is completed.

As described above, in the measurement instruments 41, 56, 61, 71, 77, 81, 91, and 121 according to the embodiments, the laser application portion 15 is configured to apply the laser beams L31 to L38 to measure the positional relationship concerning the tibia 101 or the femur 102 of the patient 100. With this configuration, the surgeon does not need to hold a heavy item, such as a measurement rod, in order to measure the positional relationship concerning the tibia 101 or the femur 102 of the patient 100. Accordingly, it is possible to reduce the burden on the surgeon when measuring the positional relationship concerning the tibia 101 or the femur 102. In addition, the laser beams L31 to L38 can be formed in a thinner line than a measurement rod. Accordingly, the laser beams L31 to L38 can be more readily and accurately applied to the leg of the patient. As a result, the surgeon can more readily perform a measurement operation using the measurement instruments 41, 56, 61, 71, 77, 81, 91, and 121.

In the measurement instruments 41, 56, 61, 71, 77, 81, 91, and 121, the laser application portion 15 is configured to apply the laser beams L31 to L38 in order to measure the positional relationships between the guide member holder portion 42, the drill 62, the valgus alignment guide 73, the spacer 82, the sizer member 92, and the guide member 122, which serve as the instruments, and the corresponding tibia 101 or the femur 102. With this configuration, in artificial knee joint implant placement surgery, the surgeon can more readily measure the positional relationships between the above respective instruments and the tibia 101 or the femur 102 of the patient.

The guide member holder portion 42, the drill 62, the valgus alignment guide 73, the spacer 82, the sizer member 92, and the guide member 122 are jigs to install an artificial knee joint implant in the body of the patient. With this configuration, in the case of, for example, temporarily installing the guide member holder portion 42, the valgus alignment guide 73, the spacer 82, the sizer member 92, and the guide member 122 that are used in artificial knee joint implant placement surgery, on the tibia 101 or the femur 102 of the patient 100, the surgeon can more readily measure the relative positions between the tibia 101 or the femur 102 of the patient 100 and the above jigs.

In the measurement instrument 41, the laser application portion 15 is configured to apply the laser beam L31 in order to measure the parallelism between the first rod 46 and the tibia 101, in a state of being supported by the guide member holder portion 42 via the connecting portion 51. With this configuration, in artificial knee joint implant placement surgery, the surgeon can more readily measure the parallelism between the first rod 46 of the guide member holder portion 42 and the tibia 101. In addition, the laser application portion 15 is installed on the guide member holder portion 42. Thus, the laser application portion 15 is held in a stable orientation by the guide member holder portion 42. Accordingly, the surgeon can more accurately measure positions using the laser beam L31 in a state where the position of the laser beam L31 is less likely to shift, unlike in the case of a configuration in which, when the surgeon uses a measurement rod instead of the laser application portion 15 and holds the measurement rod, the measurement rod position is likely to shift.

In the measurement instrument 56, The laser application portion 15 is configured to apply the laser beam L32 in order to measure the bone-cutting position in the proximal part 101a of the tibia 101. With this configuration, in artificial knee joint implant placement surgery, the surgeon can more readily and accurately measure the bone-cutting position in the proximal part 101a of the tibia 101.

In the measurement instrument 61, The laser application portion 15 is configured to apply a laser beam L33 in order to measure the coaxiality between the drill 62 and the femur 102, in a state of being supported by the drill 62 via the connecting portion 65. With this configuration, in artificial knee joint implant placement surgery, the surgeon can more readily measure the coaxiality between the distal part 102b of the femur 102 and the drill 62. In addition, the laser application portion 15 is installed in the drill 62. Thus, the laser application portion 15 is held in a stable orientation by the drill 62. As a result, the surgeon can more accurately measure positions using the laser beam L33 in a state where the position of the laser beam L33 is less likely to shift.

In the measurement instrument 71, the laser application portion 15 is configured to apply the laser beam L34 in order to measure the positional relationship between the valgus alignment guide 73 and the femur 102, in a state of being supported by the valgus alignment guide 73 via the connecting portion 76. With this configuration, in artificial knee joint implant placement surgery, the surgeon can more readily measure the positional relationship between the distal part 102b of the femur 102 and the valgus alignment guide 73. In addition, the laser application portion 15 is installed on the valgus alignment guide 73. Thus, the laser application portion 15 is held in a stable orientation by the valgus alignment guide 73. Accordingly, the surgeon can more accurately measure positions using the laser beam L34 in a state where the position of the laser beam L34 is less likely to shift, unlike in the case of a configuration in which, when the surgeon uses a measurement rod instead of the laser application portion 15 and holds the measurement rod, the measurement rod position is likely to shift.

In the measurement instrument 77, the laser application portion 15 is configured to apply the laser beam L35 in order to indicate the bone head center 102a of the femur 102, in a state of being supported by the valgus alignment guide 73 via the connecting portion 79. With this configuration, the surgeon can more readily measure the positional relationship between the bone head center 102a of the femur 102 and the valgus alignment guide 73. In addition, the laser application portion 15 is held in a stable orientation by the valgus alignment guide 73. Accordingly, the surgeon can more accurately measure positions using the laser beam L35 in a state where the position of the laser beam L35 is less likely to shift, unlike in the case of a configuration in which, when the surgeon uses a measurement rod instead of the laser application portion 15 and holds the measurement rod, the measurement rod position is likely to shift.

In the measurement instrument 81, the laser application portions 15 are configured to apply the laser beams L36 in order to indicate the bone head center 102a of the femur 102 and the leg joint center 107, in a state of being supported by the spacer 82 via the connecting portion 84. With this configuration, when checking, for example, that the knee joint center 105, the bone head center 102a of the femur 102, and the leg joint center 107 are arranged in a straight line (alignment), the surgeon can more readily measure the alignment using the laser beams L36 as marks. In addition, the laser application portions 15 are held in a stable orientation by the spacer 82. Accordingly, the surgeon can more accurately measure positions using the laser beams L36 in a state where the positions of the laser beams L36 are less likely to shift, unlike in the case of a configuration in which, when the surgeon uses a measurement rod instead of the laser application portion 15 and holds the measurement rod, the measurement rod position is likely to shift.

In the measurement instrument 91, the laser application portion 15 is configured to apply the laser beam L37 in order to measure the positional relationship between the main face 102d of the cut-bone face 102c and the sizer member 92, in a state of being supported by the sizer member 92 via the connecting portion 95. With this configuration, the surgeon can more readily measure the positional relationship between the main face 102d of the cut-bone face 102c and the sizer member 92. In addition, the laser application portion 15 is held in a stable orientation by the sizer member 92. As a result, the surgeon can more accurately measure positions using the laser beam L37 in a state where the position of the laser beam L37 is less likely to shift.

In the measurement instrument 121, the laser application portion 15 is configured to apply the laser beam L38 in order to measure the positional relationship between the guide member 122 and the distal part 102b, in a state of being supported by the guide member 122 via the connecting portion 124. With this configuration, the surgeon can more readily measure the positional relationship between the cut-bone face 102c and the guide member 122. In addition, the laser application portion 15 is held in a stable orientation by the guide member 122. As a result, the surgeon can more accurately measure positions using the laser beam L38 in a state where the position of the laser beam L38 is less likely to shift.

Although the embodiments of the present invention have been described above, the present invention is not limited to those embodiments, and various modifications may be made within the scope of claims. For example, the following modifications may be implemented.

(1) The above embodiments have been described while taking, as examples, the modes in which the laser application portion 15 is used in artificial leg joint implant placement surgery, spine correction surgery, and artificial knee joint implant placement surgery. However, this need not be the case. The laser application portion 15 may be used in surgery other than the aforementioned types of surgery.

(2) The above embodiments have been described while taking, as examples, the modes in which the laser application portion 15 is connected to the instruments using connecting portions. However, this need not be the case. The laser application portion 15 may be directly attached to the instruments.

### INDUSTRIAL APPLICABILITY

The present invention can be widely applied as a surgical measurement instrument.

### DESCRIPTIONS OF REFERENCE NUMERALS

1, 23, 41, 56, 61, 71, 77, 81, 91, 121 Surgical measurement instrument
2 Artificial leg joint implant
7 Tibia distal part cutting guide (instrument to be used in implant placement surgery/jig)
15 Laser application portion
18, 26, 51, 57, 65, 76, 79, 84, 95 Connecting portion
24 Fixed jig
31 Artificial knee joint implant
42 Guide member holder portion (instrument to be used in implant placement surgery/jig)
43 Guide member (instrument to be used in implant placement surgery/jig)
44 Clamp (distal side portion of guide member holder portion)
46 First rod (rod)
50 Spike (proximal side portion of guide member holder portion)
62 Drill (instrument to be used in implant placement surgery/jig)
72 Medullary cavity rod
73 Valgus alignment guide (instrument to be used in implant placement surgery/jig)
82 Spacer (instrument to be used in implant placement surgery/jig)
92 Sizer member (instrument to be used in implant placement surgery/jig)
101 Tibia (bone of patient)
101a Proximal part of tibia
101b Distal part of tibia
102 Femur (bone of patient)
102a Bone head center
102b Distal part of femur
103 Leg joint
105 Knee joint center
106 Knee joint
110 Spine
111 Vertebra (bone of patient)
112 Pelvis
113 Reamer hole
L1, L2, L31 to L38 Laser beam
θ1 Valgus angle direction

1. A surgical measurement instrument used in surgery to perform treatment on a bone of a patient, comprising:
a laser application portion capable of applying a laser beam for measuring a positional relationship concerning the bone.

2. The surgical measurement instrument according to claim 1,
wherein the laser application portion is configured to apply the laser beam to measure relative positions between a plurality of bones of the patient.

3. The surgical measurement instrument according to claim 1,
wherein the laser application portion is configured to apply the laser beam to measure a positional relationship between the bone of the patient and an instrument to be used in implant placement surgery to install a predetermined implant on the bone of the patient.

4. The surgical measurement instrument according to claim 3,
wherein the instrument is a jig for installing the implant in a body of the patient.

5. The surgical measurement instrument according to claim 4,
wherein the implant placement surgery includes artificial leg joint implant placement surgery for installing the implant on a leg joint including a distal part of a tibia serving as the bone of the patient,
the jig includes a tibia distal part cutting guide to be used when cutting the distal part to install the implant on the distal part, and
the laser application portion is configured to apply the laser beam toward a knee joint center of the patient, in a state of being supported by the tibia distal part cutting guide.

6. The surgical measurement instrument according to claim 2,
wherein the surgery includes spine correction surgery for correcting a spine of the patient,
the surgical measurement instrument further comprises a fixed jig that is to be fixed to a pelvis of the patient, and
the laser application portion is configured to apply the laser beam to measure a plurality of vertebrae of the spine, in a state of being supported by the fixed jig.

7. The surgical measurement instrument according to claim 4,
wherein the implant placement surgery includes artificial knee joint implant placement surgery for installing the implant on a knee joint including a distal part of a femur serving as the bone of the patient,
the jig includes a guide member holder portion for holding a guide member that guides a bone-cutting position when cutting a bone in a proximal part of a tibia of the patient,
the guide member holder portion includes a proximal side portion to be connected to the proximal part of the tibia, a distal side portion to be connected to the distal part of the tibia, and a rod for connecting the proximal side portion and the distal side portion to each other, and
the laser application portion is configured to apply the laser beam to measure parallelism between the rod and the tibia, in a state of being supported by the guide member holder portion.

8. The surgical measurement instrument according to claim 7,
wherein the laser application portion is configured to apply the laser beam to measure the bone-cutting position in the proximal part of the tibia.

9. The surgical measurement instrument according to claim 4,
wherein the implant placement surgery includes artificial knee joint implant placement surgery for installing the implant on a knee joint including a distal part of a femur serving as the bone of the patient,
the jig includes a drill for forming a reamer hole in the distal part of the femur, and
the laser application portion is configured to apply the laser beam to measure a coaxiality between the drill and the femur, in a state of being supported by the drill.

10. The surgical measurement instrument according to claim 4,
wherein the implant placement surgery includes artificial knee joint implant placement surgery for installing the implant on a knee joint including a distal part of a femur serving as the bone of the patient,
the jig includes a valgus alignment guide to be attached to the femur to guide insertion of a predetermined medullary cavity rod into a medullary cavity portion of the femur, the valgus alignment guide being for adjusting a position of the medullary cavity rod in a valgus angle direction of the femur, and
the laser application portion is configured to apply the laser beam to measure a positional relationship between the valgus alignment guide and the femur, in a state of being supported by the valgus alignment guide.

11. The surgical measurement instrument according to claim 10,
wherein the laser application portion is configured to apply the laser beam to indicate a bone head center of the femur, in a state of being supported by the valgus alignment guide.

12. The surgical measurement instrument according to claim 4,
wherein the implant placement surgery includes artificial knee joint implant placement surgery for installing the implant on a knee joint including a distal part of a femur serving as the bone of the patient,
the jig includes a spacer that is to be arranged between a cut-bone face formed in the distal part of the femur and a cut-bone face formed in a proximal part of a tibia of the patient, and
the laser application portion is configured to apply the laser beam to indicate a bone head center of the femur and a leg joint center of the patient, in a state of being supported by the spacer.

13. The surgical measurement instrument according to claim 4,
wherein the implant placement surgery includes artificial knee joint implant placement surgery for installing the implant on a knee joint including a distal part of a femur serving as the bone of the patient,
the jig includes a sizer member for positioning a pin that is to be driven into a cut-bone face formed in the distal part of the femur, and
the laser application portion is configured to apply the laser beam to measure a positional relationship between the cut-bone face and the sizer member, in a state of being supported by the sizer member.

14. The surgical measurement instrument according to claim 4,
wherein the implant placement surgery includes artificial knee joint implant placement surgery for installing the implant on a knee joint including a distal part of a femur serving as the bone of the patient,
the jig includes a guide member that is to be installed in a cut-bone face formed in the distal part of the femur, the guide member being for guiding a cutter for forming an additional cut-bone face in the distal part, and
the laser application portion is configured to apply the laser beam to measure a positional relationship between the guide member and the distal part, in a state of being supported by the guide member.

15. The surgical measurement instrument according to claim 1,
wherein the laser application portion is configured to radially apply the laser beam to the patient.
